(19) United States
(12) Patent Application Publication
(10) Pub. No.: US 2023/0121577 A1
(43) Pub. Date: Apr. 20, 2023

(54) Compositions and Methods for Treatment

(71) Applicant: The Regents of the University of California, Oakland, CA (US)

(72) Inventors: Tippi MacKenzie, San Francisco, CA (US); Tejal Ashwin Desai, San Francisco, CA (US); Stephan Sanders, San Francisco, CA (US); Xiao Huang, Gushi, Henan Province (CN); Renan B. Sper, San Francisco, CA (US)

(21) Appl. No.: 17/913,540

(22) PCT Filed: Apr. 14, 2021
(86) PCT No.: PCT/US2021/027319

§ 371 Date: Sep. 22, 2022

Related U.S. Application Data
(60) Provisional application No. 63/010,250, filed on Apr. 15, 2020.

(51) Int. Cl. A61K 31/7105; A61K 9/14; A61K 47/69

(52) U.S. Cl. CPC A61K 31/7105; A61K 9/14; A61K 47/6911; A61K 47/6937

(57) ABSTRACT

Therapeutic methods and compositions for the in utero or postnatal treatment of diseases associated with alternative splicing are provided. Compositions of the disclosure include delivery nanoparticles with an inner region surrounded by a nucleic acid scaffolding that is, in turn, linked to therapeutic agents that promote healthy mRNA splicing phenotypes in fetal cells when the compositions are delivered to a fetus in utero or in a patient after birth. The nanoparticles preferably include targeting complexes or antibodies that promote endosomal uptake into such cells and escape peptides that release the nanoparticles from endosomes into the cytosol within the cells to allow the therapeutic agents to promote preferred splicing

### CROSS-REFERENCE TO RELATED APPLICATION

This application claims benefit under 35 U.S.C. § 119(e) of Provisional Application 63/010,250, filed Apr. 15, 2020, which is hereby incorporated herein by reference in its entirety.

### BACKGROUND OF THE INVENTION

Some people suffer from disorders that are a result of alternative RNA splicing, while in other situations, alternative splicing or RNA interference could modify or treat disorders. RNA splicing is a molecular process by which a newly made precursor messenger RNA (pre-mRNA) transcript is transformed into a mature messenger RNA (mRNA). During splicing, introns (non-coding regions) are removed and exons (coding regions) are joined together into mRNA that is translated into proteins. It is understood that exons of a pre-mRNA may be spliced together in various ways to form different mRNAs in a phenomenon known as alternative splicing. Alternative splicing is regulated by activator and repressor proteins that bind to cis-acting enhancer and silencer sites on the pre-mRNA transcript to promote or reduce the usage of a particular splice site. The secondary structure of the pre-mRNA transcript also plays a role in regulating splicing, such as by bringing together splicing elements or by masking a sequence that would otherwise serve as a binding element for a splicing factor. Once example of a disease that is modified by alternative splicing is spinal muscle atrophy.

Spinal muscular atrophy (SMA) is an autosomal recessive neuromuscular disorder that leads to progressive musculoskeletal atrophy as well as bulbar and respiratory muscular weakness. It is the most common genetic cause of childhood mortality with an incidence of 1 in 11,000 live births, and a population carrier rate of nearly 1 in 50 individuals. SMA is characterized by deletions or loss-of-function mutations in the survival of motor neuron 1, telomeric (SMN1) gene on chromosome 5 (5q13.2), which encodes the protein SMN. The nearby survival of motor neuron 2, centromeric (SMN2) gene encodes a protein with the same amino acid sequence as SMN protein from SMN1. However, the protein-coding exons of SMN2 differ from SMN1 by single nucleotide in exon 7 (technically exon 8, but in standard notation referred to as exon 7 for historical reasons; this disclosure follows the standard notation). While this single nucleotide does not change the amino acids encoded (i.e., it is synonymous), it promotes alternative splicing in SMN2 leading to a higher probability of skipping exon 7, resulting in a high fraction of truncated and unstable protein (SMNΔ7). As a consequence, healthy proteins from the SMN2 gene account for about 10% of overall SMN protein function in unaffected individuals. The clinical impact of SMN1 mutations is also influenced by the number of copies of the SMN2 gene that an individual has and this varies across the population. Individuals with fewer copies of SMN2 have a more severe SMA disease phenotype. SMA type 0 often leads to fetal demise. Individuals with SMA type 1, which leads to early neonatal demise, often have two or three copies of SMN2, while types 2-4 (later onset and less severe muscular atrophy) are associated with three or more SMN2 copies. Clinical approaches to treating spinal muscle atrophy include antisense oligonucleotides (ASO), which promotes inclusion of exon 7 in the SMN2 gene, and gene therapy to replace the SMN1 gene.

Other disorders can be the consequence of reduced levels of key proteins, for example protein-truncating variants in a gene leading to nonsense-mediated decay and a reduction in functional protein or gene silencing variants. This mechanism is thought to underlie over one hundred genes associated with autism and neurodevelopmental delay. Similarly, an excess of protein can lead to disorders, for example through disordered gene regulation in Fragile X syndrome or gene duplications such as 15q11-13 that leads to neurodevelopmental delay and autism. In addition, variants can alter the function of a protein leading to a dominant negative effect (e.g. sickle cell anemia) or gain-of-function effect (e.g. infantile epileptic encephalopathy with SCN2A mutations).

### SUMMARY OF THE INVENTION

The disclosure provides therapeutic methods and compositions for the in utero or postnatal treatment of diseases associated with alternative splicing. Compositions of the disclosure include delivery nanoparticles (including those with an inner region surrounded by a nucleic acid scaffolding) linked to therapeutic agents that promote advantageous mRNA splicing phenotypes in cells when the compositions are delivered to a fetus in utero or after birth. The nanoparticles preferably include targeting complexes such as antibodies that promote endosomal uptake into such cells and escape peptides that release the nanoparticles from endosomes into the cytosol within the cells to allow the therapeutic agents to promote advantageous splicing.

The disclosure provides methods and compositions for in utero or postnatal gene therapy using modular multifunctional nanoparticles. Methods and compositions of the disclosure have the potential to treat severe or fatal disorders such as thalassemias, lysosomal storage disorders, or genetic causes of developmental delay. The fetal environment offers the potential advantages of accessing stem cells during a unique time window of proliferation and migration, inducing tolerance to new proteins, as well as correcting neurologic diseases before the blood brain barrier closes, and requiring lower doses than postnatal therapy due to the lower body weight.

Methods and compositions of the disclosure use biocompatible synthetic nanoparticles (NPs) as a transient delivery strategy for treatments that can include antisense oligonucleotides, gene therapies, and gene editing reagents such as Cas9 and guide RNA (gRNA). Delivery with nanoparticles of the disclosure may be safer and more controllable that viral vectors such as AAV9. In preferred embodiments, methods and compositions of the disclosure use DNA-coated NPs to deliver gene therapy and related treatments. The nanoparticles include an inner region surrounded by a nucleic acid scaffold that protects and aids delivery of the particle. Results show that these DNA-NPs can be injected safely to fetuses in utero and are internalized by hematopoietic and stromal cells.

Methods and compositions of the disclosure include a modular delivery platform using nanoparticles made of biocompatible and biodegradable polymer along with a surface scaffold of short synthetic DNA to facilitate the loading of functional biomolecules such as therapeutic agents, targeting antibodies and endosomal escape enhancers. DNA hybridization-guided assembly of biomolecules results in dramatically improved efficiency of surface loading compared with prior art nanoparticles. The loading amount of oligonucleotide/peptide cargo on the surface is higher than that loaded in the core through double-emulsion protocol, promoting a surface-scaffolding of DNA in the particles (rather than burying such DNA in the core).

Using short synthetic DNA as surface scaffolds allows the co-loading of multiple bioreactive cargos, and the precise ratiometric control of each moiety, thus providing a fast and efficient method of NP functionalization. Furthermore, the assembly of high molecular weight moieties (e.g., antibodies and other proteins) above a specific density can protect the DNA scaffolds and associated nucleic acid therapies such as plasmids or antisense oligonucleotides from enzymatic degradation by steric inhibition. This nanoparticle delivery platform is particularly amenable to the delivery of ASOs for the treatment of diseases involving alternative splicing, such as spinal muscular atrophy (SMA), Angelman syndrome, or muscular dystrophy, or the reduction of gene expression through gapmer-ASOs, for example to decrease the expression of a gain-of-function missense mutation.

Gene therapy and nucleotide therapies of the disclosure may be used to modify disorders associated with disordered gene regulation (e.g., Fragile X syndrome) gene duplications (e.g., 15q11-13 that leads to neurodevelopmental delay and autism), variants that alter the function of a protein leading to a dominant negative effect (e.g., sickle cell anemia) or gain-of-function effect (e.g., infantile epileptic encephalopathy with SCN2A mutations). Compositions and methods of the disclosure may operate through gene replacement, gene editing (e.g., CRISPR), or modifying the degree of gene expression (e.g., gapmer-ASOs that promote RNA degradation or splice altering ASOs to convert noncoding transcripts into protein-coding transcripts).

Nanoparticles may be introduced into a developing fetus as a delivery system for a missing protein, e.g., as a continuous delivery system, with the purpose of inducing tolerance to that protein. For example, fetal infusion of a lysosomal enzyme can induce tolerance to this missing protein in fetuses with lysosomal storage disorders. Infusions of a clotting factor can induce tolerance in a fetus with hemophilia. Although the recombinant proteins could be infused alone, putting these in a nanoparticle or a comparable delivery vehicle will prolong the half-life of these proteins and improve the process of fetal tolerance induction. The fetus required continuous exposure to become tolerant (the infused protein or peptides can better access the thymus and be presented to developing fetal T cells for deletion of T cells that are reactive to these proteins (which is the mechanism required to induce tolerance).

Nanoparticles may be used to deliver small RNA particles such as a short hairpin RNA (shRNA), including those that can be designed to alter gene expression, such as knocking down a repressor to increase expression of a target. For example, shRNA may be used to knockdown BCL11a to induce fetal hemoglobin. Other short oligonucleotides could be delivered for RNA interference to modify gene expression. Therapies that employ small RNA particles can be selectively delivered by targeting the carrier nanoparticle to a specific cell type. For example, hematopoietic cells may be targeted using an antibody or antigen receptor moiety with the nanoparticle.

Preferred embodiments use nanoparticles coated with nucleic acid scaffolding that can bind to as ASO or RNA; the sequence of the nucleic acids on the NP can be modified for tuning the timing of release of the therapeutic ASO or RNA molecule, since the therapeutic molecules can be released more quickly if there is less sequence homology between the nucleic acid scaffold moiety on the NP and the therapeutic molecule.

In certain aspects, the disclosure provides a composition for treating a disorder. The composition includes a nanoparticle and an antisense oligonucleotide carried by the nanoparticle. Preferably, the antisense oligonucleotide is complementary to, and hybridizes to, a messenger RNA (mRNA) (e.g., from a survival motor neuron gene) when delivered to a fetal cell in utero. The antisense oligonucleotide may be a splice-switching oligonucleotide (SSO) or a gapmer-ASO. The nanoparticle may include a plurality of targeting complexes, e.g., coating an exterior surface of the nanoparticle. The complexes may be, for example, antibodies that bind the nanoparticle to cell-surface markers that are specific to fetal cells, specific to neurons, or both.

The antisense oligonucleotides may be between about 10 and 35 nucleotides in length, preferably between about 15 and 30. One or more nucleotides in the antisense oligonucleotide may include a modification to prevent degradation and promote binding to the target RNA, such as base methylation; phosphorothiate backbone modification; 2′-O-methyl; 2′-O-methoxyethyl; locked nucleic acid (LNA); or phosphorodiamidate morpholinos (PMOs).

In certain embodiments, the antisense oligonucleotide is complementary to, and hybridizes to, an mRNA from a gene selected from the group consisting of: survival motor neuron 1, survival motor neuron 2, β-globin, the IKBKAP gene, the DMD gene, the UBE3A-ATS gene, the SCN2A gene, the SCN8A gene, the SCN3A gene, and genes for developmental disorders.

In some embodiments, the composition includes an antisense oligonucleotide that is provided by a process that includes sequencing a nucleic acid from the fetus to obtain genetic information, identifying a splicing variant in the genetic information, designing the antisense oligonucleotide to target the identified splicing variant, and synthesizing or obtaining the designed antisense oligonucleotide. Compositions and methods of the disclosure may be used to address disorders or genes that are not listed here or not yet known, e.g., in the literature. Methods of the disclosure may include genetic sequencing of a fetus to reveal a splice variant, targeting an antisense oligonucleotide to the newly revealed splice variant, packaging the antisense oligonucleotide in a nanoparticle of the disclosure, and providing the nanoparticle(s) for therapeutic use in in utero or postnatal delivery.

The nanoparticle may be composed such that an inner region of the nanoparticle comprises a polymer (e.g., poly lactic-co-glycolic acid), metal (e.g., gold or silver), or a liposome, and an outer region of the nanoparticle comprises a scaffold of nucleic acid. The antisense oligonucleotide may be linked to the nucleic acid of the scaffold. The nanoparticle may also include one or a plurality of endosomal escape peptides linked to the nucleic acid of the scaffold, a plurality of targeting complexes linked to the nucleic acid of the scaffold, or both. The targeting complexes may be antibodies that bind to cell-surface markers on fetal cells. The antisense oligonucleotides may be linked to the nucleic acid of the scaffold by disulfide bonds. The inner region of the nanoparticle may surround a core that contains a payload such as a small molecule, a protein, or a nucleic acid.

Some embodiments may use an antibody against the Ckit receptor on hematopoietic stem cells to deliver nanoparticles directly into that cell type. The disclosure includes data that show the ability to target Ckit+ cells in mouse fetal liver, as well as DLK-1 positive hepatocyte precursors or CD45+ leukocytes.

In preferred embodiments, the antisense oligonucleotide is a splice-switching oligonucleotide (SSO) complementary to an mRNA from a gene for which alternative splicing is associated with a disease such as, for example, a survival motor neuron (SMN) gene. Preferably, when the nanoparticle is injected into circulation in a fetus: the targeting complexes target the nanoparticles to neurons or precursors thereof; the endosomal escape peptides cause release of the nanoparticles into cytosol of the neurons or the precursors thereof; the SSO is released from the nanoparticle upon exposure to glutathione in the cytosol; and the SSO binds to an SMN mRNA and prevents formation of an isoform associated with spinal muscle atrophy.

In some certain embodiments, an inner region of the nanoparticle comprises PLGA and an outer region of the nanoparticle comprises a scaffold of nucleic acid linked to the antisense oligonucleotides, targeting complexes, and, optionally, endosomal escape peptides, in which the antisense oligonucleotide is a splice-switching oligonucleotide (SSO) complementary to an mRNA from a gene associated with a disease, such that, when the nanoparticle is injected into circulation in a fetus: the targeting complexes target the nanoparticles to fetal cells preferably of a specific type; the escape peptides—if included—cause release of the nanoparticles into cytosol of the cells; the SSO is released from the nanoparticle into the cytosol; and the SSO binds to an mRNA and prevents formation of splicing of the mRNA into a disease-associated isoform or promotes splicing in a manner advantageous to health.

Other aspects of the disclosure provide a composition that includes gene-editing reagents, or nucleic acid encoding the gene-editing reagents. The gene-editing reagents are targeted to a gene for which a variant promotes an alternative splicing of mRNA that causes a disease, such that when the composition is delivered to a fetus in utero, the gene editing reagents correct or ameliorate the effect of the variant to thereby inhibit the alternative splicing. The gene-editing reagents (or nucleic acid encoding them) are preferably packaged for delivery with a nanoparticle. In certain embodiments, an inner region of the nanoparticle comprises a polymer, metal, or liposome, and an outer region of the nanoparticle comprises a scaffold of nucleic acid. A plurality of targeting complexes such as antibodies may be linked to the nanoparticle. The nanoparticle may also be decorated with endosomal escape peptides. The gene editing reagents include zinc-finger nucleases (ZFNs), transcription activator-like effector nucleases (TALENs), peptide-nucleic acids (PNAs), or a CRISPR system. For example, in some embodiments, the nanoparticle includes the nucleic acids encoding the gene editing reagents and the gene editing reagents include a Cas endonuclease. In certain embodiments, the gene editing reagents include a Cas endonuclease complexed with a guide RNA as a ribonucleoprotein (RNP) within the nanoparticle. Preferably, the guide RNA targets the Cas endonuclease to a survival motor neuron gene to inhibit alternative splicing of the SMN gene to inhibit spinal muscle atrophy in the fetus.

In certain aspects, the disclosure provides a therapeutic composition that includes a nanoparticle; and a therapeutic agent carried by or on the nanoparticle. The therapeutic agent may be a combination of small molecules, nucleotide sequences, and/or proteins. For example, the therapeutic agent may include one or more nucleotide sequences. In some embodiments, the therapeutic agent is one or more antisense oligonucleotides (ASO). In one illustrative embodiment, the antisense oligonucleotides are between about 10 and 35 nucleotides in length; and one or more nucleotides in the antisense oligonucleotide includes one or more modifications to prevent degradation, improve RNA binding efficiency, and/or reduce toxicity, the modification selected from the group including: base methylation; phosphorothiate (PS) backbone modification; 2′-O-methyl (2′-OMe); 2′-O-methoxyethyl (2′-MOE); locked nucleic acid (LNA); and phosphorodiamidate morpholinos (PMOs).

Alternatively or additionally, the therapeutic agent may include one or more splice-switching oligonucleotides (SSO); short hairpin RNAs (shRNA); small interfering RNAs (siRNA); or combinations thereof. The therapeutic agent may be designed to induce immune tolerance when delivered in utero or after birth.

In some embodiments, the therapeutic agent is one or more nucleic acids comprising nucleotide sequences complementary to the RNA of a gene selected from the group consisting of survival motor neuron 1, survival motor neuron 2, β-globin, blc11a, the IKBKAP gene, the DMD gene, the UBE3A gene, the UBE3A-ATS gene, the SCN2A gene, the SCN8A gene, the SCN3A gene, and genes for other developmental disorders. The therapeutic agent may include one or more nucleic acids comprising nucleotide sequences complementary to the RNA of a gene with multiple isoforms occurring physiologically with the objective of increasing the proportion of transcripts containing specific exons. Preferably the therapeutic agent is one or more nucleic acids comprising nucleotide sequences complementary to the RNA of a gene in which a genetic variant disrupts physiological splicing with the objective of restoring normal splicing behavior. The therapeutic agent may include one or more nucleic acids comprising nucleotide sequences complementary to the RNA of a gene in which a genetic variant leads to an encoded protein that has a gain-of-function or dominant negative effect with the objective of decreasing the quantity of the abnormal RNA or encoded protein. The therapeutic agent may include one or more nucleotide sequences complementary to the genome of a pathogen.

In some embodiments, the nanoparticle further comprises a plurality of targeting complexes. The composition may include one or a plurality of endosomal escape peptides linked to the nucleic acid of the scaffold. The composition may include reagents to facilitate passage across the blood-brain barrier.

In certain embodiments, an inner region of the nanoparticle comprises a polymer, metal, or liposome, and an outer region of the nanoparticle comprises a scaffold of nucleic acid. The composition may include a plurality of targeting complexes linked to the nucleic acid of the scaffold (e.g., antibodies that bind to cell-surface markers on fetal cells). In some embodiments, the inner region comprises the polymer, poly lactic-co-glycolic acid (PLGA). The inner region of the nanoparticle may surround a core that contains a payload. The payload may include one or more of a small molecule, a protein, and a nucleotide sequence.

One or more nucleotide sequences may be carried within the inner region or core of the nanoparticle. One or more nucleotide sequences may be linked to the nucleic acid of the scaffold.

In various optional embodiments: the nucleotide sequences are splice-switching oligonucleotides (SSOs) complementary to RNA from a survival motor neuron (SMN) gene; the nucleotide sequences are splice-switching oligonucleotides (SSOs) complementary to RNA from the DMD gene; or the nucleotide sequences are antisense oligonucleotides (ASOs) complementary to an antisense RNA or IncRNA including the UBE3A-ATS gene or XIST gene.

The one or more nucleotide sequences may be linked to the nucleic acid of the scaffold by disulfide bonds.

The composition may include a combination of nucleotide sequences and proteins, including targeting antibodies and/or escape peptides, linked to the nucleic acid of the scaffold.

The composition may include a combination of small molecules, nucleotide sequences and proteins, including targeting antibodies and/or escape peptides, linked to the nucleic acid of the scaffold or contained within the inner region or core of the nanoparticle. The scaffold of nucleic acid may contain multiple nucleotide sequences of varying length and varying degrees of complementarity to a therapeutic nucleotide sequence.

In some embodiments, when the nanoparticle is injected into circulation before or after birth: the targeting complexes target the nanoparticles to neurons or precursors thereof; the endosomal escape peptides cause release of the nanoparticles into cytosol of the neurons or the precursors thereof; the nucleotide sequences are released from the nanoparticle upon exposure to glutathione in the cytosol; and the nucleotide sequences bind to RNA to modify splicing or degrade the RNA. In certain embodiments, an inner region of the nanoparticle comprises a PLGA and an outer region of the nanoparticle comprises a scaffold of nucleic acid linked to nucleotide sequences, targeting complexes, and endosomal escape peptides, wherein the nucleotide sequences are complementary to an RNA from a gene associated with a disease, wherein, when the nanoparticle is injected into circulation in a fetus: the targeting complexes target the nanoparticles to cells of a specific type; the escape peptides cause release of the nanoparticles into cytosol of the cells; the nucleotide sequences are released from the nanoparticle into the cytosol; and the nucleotide sequences bind to RNA to modify splicing or degrade the RNA. It may be that the therapeutic agent includes splice-switching oligonucleotides (SSO) complementary to an SMN gene RNA and induce the generation of isoforms that produce stable and functional protein to treat spinal muscular atrophy. Optionally the therapeutic agent includes splice-switching oligonucleotides (SSO) complementary to genes in which a stop codon leads to degradation of the RNA of one or more isoforms with the intent to increase expression.

The nucleotide sequences may have a sequence that targets a specific gene and in which: the gene to be targeted is DMD to skip exons with genetic variants leading to muscular dystrophy; or the gene to be targeted is SCN1A to skip exons that would lead to nonsense-mediated decay as a treatment for Dravet syndrome. In certain embodiments the nucleotide sequences are antisense oligonucleotides (ASOs) complementary to the UBE3A-ATS RNA leading to its degradation resulting in upregulation of the UBE3A gene to treat Angelman syndrome.

Aspects of the disclosure relate to a composition that includes gene-editing reagents, or nucleotide sequences encoding the gene-editing reagents, wherein the gene-editing reagents are targeted to a gene for which a variant contributes to a disease, such that when the composition is delivered to a fetus in utero, the gene editing reagents correct or ameliorate the effect of the variant; or gene-editing reagents, or nucleotide sequences encoding the gene-editing reagents, wherein the gene-editing reagents are targeted to a gene that modifies a disease process, such that when the composition is delivered to a fetus in utero, the gene editing reagents correct or ameliorate the disease process; or reagents targeted to the cis regulatory regions of a gene, or nucleotide sequences encoding the reagents, wherein the reagents are targeted to a gene for which a variant causes a disease process, such that when the composition is delivered to a fetus in utero, the reagents correct or ameliorate the disease process by increasing gene expression, decreasing gene expression or modifying splicing and isoform usage.

In preferred embodiments, the reagents, or the nucleotide sequences encoding the reagents, are packaged with a nanoparticle for delivery. In some embodiments, an inner region of the nanoparticle comprises a polymer (e.g., PLGA), a metal (e.g., gold or silver), or a liposome, and an outer region of the nanoparticle comprises a scaffold of nucleic acid. The composition may include a plurality of targeting complexes linked to the nanoparticle. The composition may include endosomal escape peptides linked to the nanoparticle. The gene editing reagents may include zinc-finger nucleases (ZFNs), transcription activator-like effector nucleases (TALENs), peptide-nucleic acids (PNAs), or Cas endonuclease.

In some embodiments, the nanoparticle includes nucleotide sequences encoding the gene editing reagents and the gene editing reagents include a Cas endonuclease. Preferably the gene editing reagents include a Cas endonuclease complexed with a guide RNA as a ribonucleoprotein (RNP) within the nanoparticle. In some embodiments, the guide RNA targets the Cas endonuclease to a survival motor neuron gene to modify splicing of the SMN gene to produce stable and functional SMN protein to treat spinal muscle atrophy in the fetus. In other embodiments, the guide RNA targets the Cas endonuclease to the CFTR gene to produce stable and functional CFTR protein to treat cystic fibrosis in the fetus. The nanoparticle may include nucleotide sequences encoding a gene to replace one or more copies that are defective leading to disease in an individual. For example, the gene to be replaced may be: CFTR, HBB, SERPINA1, SLC26A4, KCNJ10, GALNS, DMD, F8, F9, F9, HBA2, HBA1, FMR1, HGSNAT, SFTPB, SGSH, SMN1, GBA, or SCARB2.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 shows in utero therapy provides advantages in genetic disorders. Prenatal or postnatal treatment of genetic disorders has many advantages, including (1) early treatment before irreversible pathology (2) higher accessibility of progenitor cells with local therapeutic administration (3) more tolerated environment with less immune rejection to therapeutics than systemic administration.

FIG. 2 shows use of multi-functional nanocarriers to facilitate intracellular delivery. Most of the gene regulators with high therapeutic development interest, including antisense oligo, small interfering RNA, mRNA, and CRISPR/Cas9, need to get internalized by a small population of cells specifically. However, the delivery of these biomolecules with large molecular weight and high hydrophilicity is challenging due to cellular membrane barriers and systemic clearance. Nanocarriers can provide an excellent protection for these fragile biomolecules and promote efficient and targeted delivery into intended cell populations. Using our novel platform of DNA-scaffolded nanoparticles for efficient and versatile surface functionalization, we can well organize three moieties essential for intracellular delivery: 1) cell targeting module, 2) endosomal escape module, and 3) glutathione cleavable gene regulatory module.

FIG. 3 shows an α-c-kit antibody coating of nanoparticles targeting C-kit⁺ cells ex vivo. An anti-C-kit antibody is conjugated with modified DNA that is complementary to scaffolds on nanoparticles so that the surface density of the antibody can be precisely controlled. Nanoparticles coated with anti-C-kit antibody through DNA hybridization show specific association with C-kit positive cells after ex vivo co-incubation for 1 hour, compared to nanoparticles without anti-C-kit antibody coating.

FIG. 4 shows that α-c-kit antibody coated nanoparticles target HSCs ex vivo. Murine hematopoietic stem cells (Lin- Ckit+ Sca1+) harvested from fetal livers show higher association with anti-C-kit-coated nanoparticles (AF647 in core) after ex vivo co-incubation, than with nanoparticles without anti-C-kit antibody. It is interesting that C-kit receptor expression level in Sca-1+ cells is decreased at 2 hours post nanoparticle co-incubation, but the expression level is recovered after 48 hours.

FIG. 5 shows that α-c-kit antibody coated nanoparticles also target Lin⁺C-kit⁺ cells ex vivo. Anti-C-kit antibody coated nanoparticles also target Lin+ C-kit+ cell subsets isolated from fetal livers, which are differentiation derivatives of HSCs.

FIG. 6 shows α-c-kit-nanoparticles are internalized by cells ex vivo. Harvested cells from fetal livers are co-incubated with multifunctional nanoparticles that are coated with anti-C-kit antibody, endosomal escaping peptide GALA, and antisense oligos for 2 hours, and cells that are associated with particles are sorted through flow sorter. Sorted cells are imaged through spinning disk confocal microscope at different z-stacks. Images show that nanoparticles are internalized into cells, and GALA peptide coating shows signs of cytosolic release of ASO on nanoparticles.

FIG. 7 shows NP: AF647 (core) ASO+(AF555) Ckit+ GALA+(FITC) and NP: AF647 (core) ASO+(AF555) Ckit+ no GALA.

FIG. 8 shows NP: AF647 (core) ASO+(AF555) no Ckit no GALA. AF647+ cell population was sorted and imaged by a SD confocal 100× EMCCD camera.

FIGS. 9A-9B show the biodistribution between IV and IU injection. In utero injection of anti-C-kit coated nanoparticles shows liver localization in fetus. FIG. 9A shows immunofluorescence images after in utero injection into fetal mice at 10 OD concentration of nanoparticles, imaged at 40× power. The images show DAPI signal (seen in nuclei), the Cy5.5 dye carried by the nanoparticle (indicating nanoparticle localization inside these cells), as well as CD71 receptor on fetal cells. The “overlay” images show these colors together at 40× or 10×. A cartoon mouse is drawn to show the position of the fetuses where fetus 8 is the 8^{th} fetus counting from the mouse’s right side. FIG. 9B shows an immunofluorescent microscope image of a fetus after in utero injection of nanoparticles carrying the Cy5.5 dye, showing presence of nanoparticles in the regions of the thorax and liver as indicated.

FIG. 10 shows α-c-Kit Ab coated nanoparticles accumulate more in Lin⁻ cells in vivo. Utero injection of nanoparticles that are coated with anti-C-Kit antibodies are internalized more by lineage negative cell subsets than control particles without anti-C-kit antibody after 5 hours.

FIGS. 11A-11E show that nanoparticles coated with an antibody against CD45 show specific association with CD45+ cell subsets after 1 hour incubation with hematopoietic cells harvested from mouse fetal liver. CD45+ cell subsets were targeted using nanoparticles coated with anti-CD45 antibodies. High-density DNA-streptavidin-CD45 - Quasar705 (surface) analysis was performed 1 hour post incubation using flow cytometry. The plots demonstrate the gating scheme to detect nanoparticle (visualized using the q705 dye on the NP). FIG. 11A shows that live cells are separated into CD45+ gates. FIG. 11B shows the amount of q705 dye (i.e., nanoparticle carried by CD45+ cells from 3 experiments (1. No particle; 2. NP carrying a CD45 antibody; 3. NP not carrying an antibody) as an overlay, demonstrating the increase in the amount of q705 (and therefore nanoparticle) in CD45+ cells when the nanoparticle is also labeled with a CD45 antibody. 0.05 OD refers to the concentration of nanoparticle used in this experiment. FIG. 11C is a that plot demonstrates the q705 dye carried in the population of cells that are CD45+ and Ter119+. FIG. 11D depicts the percentage of CD45+ cells that are positive for the q705 dye in the experimental conditions: no nanoparticle; NP labeled with CD45+ antibody or isotype control at 0.05 or 0.5 OD, demonstrating higher labeling when the NP carries the CD45 antibody at the higher concentration. FIG. 11E demonstrates the percentage of CD45+ Ter119+ cells at the same experimental conditions, demonstrating higher labeling of these cells at the higher concentration of CD45-coated nanoparticles.

FIGS. 12A-12E show that nanoparticles coated with an antibody against DLK1 show specific association with DLK1+ cell subsets (a marker for fetal hepatic stem cells) after 1 hour incubation with cells harvested from mouse fetal liver. FIG. 12A shows the gating scheme for DLK1+ cells. FIG. 12B shows the next gate for detection of q705 dye contained in the nanoparticles. FIG. 12C is the graphed data for separate experimental conditions (no nanoparticle; DLK1- coated NP or isotype control-coated NP at 0.05 OD or 0.5 OD concentration), demonstrating specificity when the NP carries the DLK1-antibody. FIG. 12D shows gating on DLK1-negative cells, and FIG. 12E shows the graph of these data in the experimental conditions indicated, demonstrating relative lack of NP inside these cells compared to DLK1+ cells in FIG. 12C.

FIG. 13 shows this platform can be adapted for various biomolecule delivery.

FIG. 14 shows a nanoparticle composition for treating a disease.

FIG. 15 shows features that may be included in a nanoparticle of the disclosure.

FIG. 16 shows a schematic for in utero treatment of an alternative splicing disease.

FIG. 17 diagrams knock-in to add a sequence to a gene via homology directed repair.

FIG. 18 shows gels from PCR amplification after CRISPR knock-in into a gene.

FIG. 19 shows fluorescent images of cells after CRISPR knock-in into a gene.

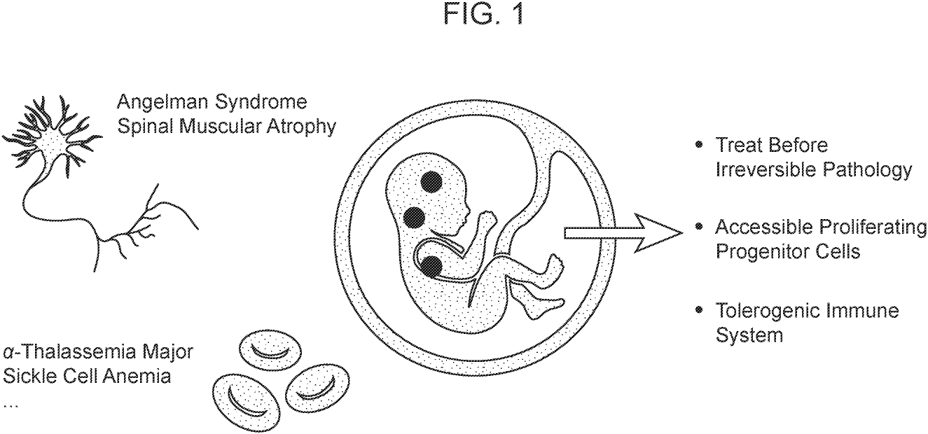

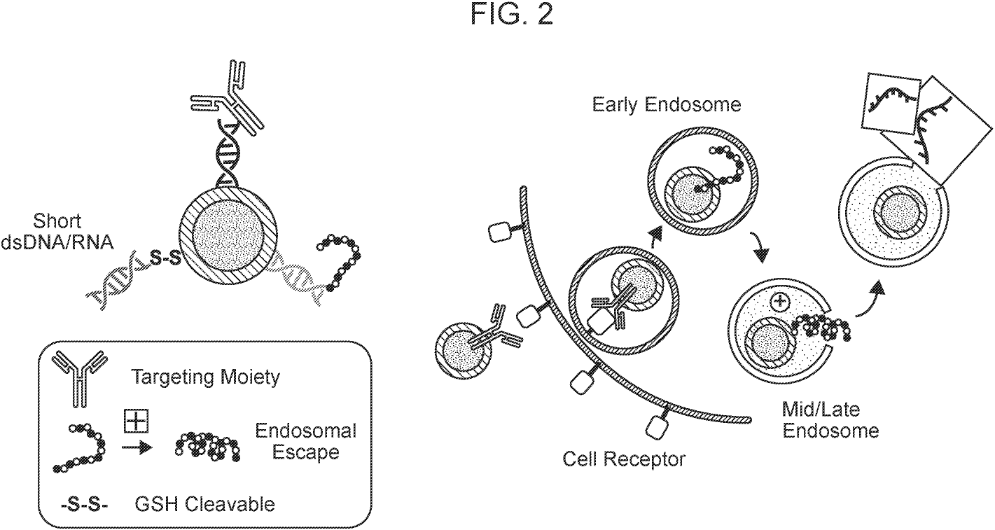

### DETAILED DESCRIPTION OF THE INVENTION

Methods and compositions for the delivery of therapeutic agents such as antisense oligonucleotides, small interfering RNAs (siRNAs), short hairpin RNAs (shRNAs), gene therapy agents, proteins, small molecules, gene editing agents such as CRISPR systems (gRNA/Cas9), and other such materials are provided for the in utero or postnatal treatment of diseases associated with alternative splicing. Compositions of the disclosure include nanoparticles for delivery of therapeutics, including those with nucleic acid scaffolding linked to a therapeutic agent that promote advantageous mRNA splicing phenotypes in cells when the compositions are delivered to a fetus in utero or after birth. Nanoparticles may be used to deliver small RNA particles such as siRNA or shRNA, including those that can be designed to alter gene expression, such as knocking down a repressor to increase expression of a target. Nanoparticle-based methods and compositions of the disclosure may also be used to deliver a protein, such as a missing protein not being naturally expressed by a subject or a CRISPR system to genetically modify a cell to eliminate a disease-causing mutation associated with alternative splicing.

Before the present methods and compositions are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof, e.g., polypeptides known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about,” particularly in reference to a given quantity, is meant to encompass deviations of plus or minus five percent.

By “diseases associated with alternative splicing” is meant any disease associated with aberrant splicing of RNA. Alternative splicing may result, for example, in reduced levels of a protein, truncated protein variants, a reduction in functional protein, gene silencing variants, instability of a protein, disordered gene regulation, or variants that alter the function of a protein. Diseases associated with alternative splicing include, but are not limited to, spinal muscular atrophy, Angelman syndrome, myotonic dystrophy, Duchenne muscular dystrophy (DMD), choroideremia, Pompe disease, spinocerebellar ataxia, beta thalassemia, cancer, inflammatory conditions, Frasier syndrome, frontotemporal dementia and Parkinsonism linked to chromosome 17 (FTDP-17), atypical cystic fibrosis, diabetes, Usher syndrome, sickle cell anemia, thalassemias, Fanconi’s anemia, familial dysautonomia, Hutchinson-Gilford progeria syndrome (HGPS), hyperchole sterolemia, Prader-Willi syndrome, medium-chain acyl-CoA dehydrogenase (MCAD) deficiency, tauopathies, lysosomal storage disorders neurodevelopmental delay disorders, and metabolic disorders.

By “subject” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

“Diameter” as used in reference to a shaped structure (e.g., nanoparticle, pore, cell, cell aggregate, etc.) refers to a length that is representative of the overall size of the structure. The length may in general be approximated by the diameter of a circle or sphere that circumscribes the structure.

By “nanoparticle” is meant a particle having at least one dimension (e.g., a greatest dimension) in the range of from 1 nanometer (nm) to 1000 nm, from 20 nm to 750 nm, from 50 nm to 500 nm, including from 100 nm to 300 nm. The nanoparticle may have any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. In certain embodiments, the nanoparticle (e.g., a spherical or spheroid particle) has a greatest dimension of from 10 to 200 nm, e.g., from 30 to 100 nm. According to some embodiments, the greatest dimension of the nanoparticle (e.g., the diameter in the case of a spherical or spheroid nanoparticle) is greater than 10 nm but 500 nm or less, 450 nm or less, 400 nm or less, 350 nm or less, 300 nm or less, 250 nm or less, 200 nm or less, or 100 nm or less. In certain embodiments, the greatest dimension of the nanoparticle (e.g., the diameter in the case of a spherical or spheroid nanoparticle) is less than 500 nm, but 10 nm or greater, 20 nm or greater, 30 nm or greater, 40 nm or greater, 50 nm or greater, 60 nm or greater, 70 nm or greater, 80 nm or greater, 90 nm or greater, 100 nm or greater, 125 nm or greater, 150 nm or greater, 175 nm or greater, 200 nm or greater, 225 nm or greater, 250 nm or greater, 275 nm or greater, 300 nm or greater, 350 nm or greater, or 400 nm or greater.

The nanoparticle may be made of any suitable material or mixtures thereof. Suitable materials include, but are not limited to, organic or inorganic polymers, natural and synthetic polymers, including, but not limited to, agarose, cellulose, nitrocellulose, cellulose acetate, other cellulose derivatives, dextran, dextran-derivatives and dextran co-polymers, other polysaccharides, glass, silica gels, gelatin, polyvinyl pyrrolidone, rayon, nylon, polyethylene, polypropylene, polybutylene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamides, polyacrylamide blends, co-polymers of vinyl and acrylamide, methacrylates, methacrylate derivatives and co-polymers, polyethylene glycol, polylactic acid, poly(lactic-co-glycolic acid), polycaprolactone, other polymers and co-polymers with various functional groups, latex, butyl rubber and other synthetic rubbers, silicon, glass, insoluble protein, metals and metal oxides (e.g., gold, silver, nickel, platinum, iron oxide, titanium dioxide, and the like), metal alloys, metalloids, magnetic materials, and any combinations thereof. The nanoparticles may be magnetically responsive, e.g., by virtue of comprising one or more paramagnetic and/or superparamagnetic substances, such as for example, magnetite. Such paramagnetic and/or superparamagnetic substances may be embedded within a matrix of the nanoparticle, and/or may be disposed on an external and/or internal surface of the nanoparticle. Nanoparticles may also include liposomes, lipid nanoparticles, solid lipid nanoparticles, and lipid-polymer hybrid nanoparticles. Such nanoparticles may comprise, without limitation, cationic lipids (e.g., 1,2-dioleoyl-sn-glycero-3-ethylphosphocholine, didodecyldimethylammonium bromide (DDAB), 3-dioleyloxy)propyl]-N,N,N-trimethylammonium chloride (DOTMA), 1,2-dioleyloxy-3-dimethylaminopropane (DODMA), 1, 2-oleinyl-3-trimethylammonium-propane (DOTAP), N-[1-(2,3-oleoyloxy)propyl]-N,N,N-trimethyl-ammonium methyl sulfate, dioleoylphosphatidyl ethanolamine (DOPE), oleic acid (OA), dimethyldioctadecyl ammonium bromide, N4-cholesteryl-spermine (GL67), and the like), phospholipids (e.g., phosphatidylcholine, phosphatidylethanolamine), sterols (e.g., cholesterol), PEGylated lipids, triglycerides (e.g. tristearin), diglycerides (e.g. glycerol bahenate), monoglycerides (e.g. glycerol monostearate), fatty acids (e.g. stearic acid), waxes (e.g. cetyl palmitate), sphingomyelins, bile salts (sodium taurocholate), or surfactants, and any combinations thereof.

By “isolated” is meant, when referring to a polypeptide or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The terms “treat”, “treating”, treatment,” “prevent,” “preventing,” and the like refer to a course of action (such as administering an agent or a pharmaceutical composition comprising an agent) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, or condition afflicting a subject. Thus, treatment includes inhibiting (i.e., arresting the development or further development of the disease, disorder or condition or clinical symptoms association therewith) an active disease.

The term “in need of treatment” as used herein refers to a judgment made by a physician or other caregiver that a subject requires or will benefit from treatment. This judgment is made based on a variety of factors that are in the realm of the physician’s or caregiver’s expertise.

The phrase “therapeutically effective amount” refers to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The term “antibody” encompasses monoclonal antibodies as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N— or C—glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, microRNA, DNA:RNA hybrids, and hybrids between PNAs and DNA or RNA, and also include known types of modifications, for example, labels which are known in the art, methylation, “caps,” substitution of one or more of the naturally occurring nucleotides with an analog (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30: 1911-1918; Elayadi et al. (2001) Curr. Opinion Invest. Drugs 2: 558-561; Orum et al. (2001) Curr. Opinion Mol. Ther. 3: 239-243; Koshkin et al. (1998) Tetrahedron 54: 3607-3630; Obika et al. (1998) Tetrahedron Lett. 39: 5401-5404.

The terms “hybridize” and “hybridization” refer to the formation of complexes between nucleotide sequences which are sufficiently complementary to form complexes via Watson-Crick base pairing.

The term “hybridization conditions” as used herein refers to conditions that allow hybridization of a nucleic acid to a complementary nucleic acid, e.g., a nucleic acid immobilized in a scaffold on a nanoparticle may specifically bind to a complementary nucleic acid via Watson-Crick base pairing under hybridization conditions.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequences. Homologous regions may vary in length, but will typically be between 4 and 500 nucleotides (e.g., from about 4 to about 40, from about 40 to about 80, from about 80 to about 120, from about 120 to about 160, from about 160 to about 200, from about 200 to about 240, from about 240 to about 280, from about 280 to about 320, from about 320 to about 360, from about 360 to about 400, from about 400 to about 440, etc.).

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when a uracil is denoted in the context of the present invention, the ability to substitute a thymine is implied, unless otherwise stated. “Complementarity” may exist between two RNA strands, two DNA strands, or between a RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

A “target site” or “target sequence” is the nucleic acid sequence recognized (i.e., sufficiently complementary for hybridization) by a therapeutic agent, a guide RNA (gRNA), or a homology arm of a donor polynucleotide. The target site may be allele-specific (e.g., a major or minor allele).

The term “donor polynucleotide” refers to a polynucleotide that provides a sequence of an intended edit to be integrated into the genome at a target locus by homology-directed repair (HDR).

By “homology arm” is meant a portion of a donor polynucleotide that is responsible for targeting the donor polynucleotide to the genomic sequence to be edited in a cell. The donor polynucleotide typically comprises a 5′ homology arm that hybridizes to a 5′ genomic target sequence and a 3′ homology arm that hybridizes to a 3′ genomic target sequence flanking a nucleotide sequence comprising the intended edit to the genomic DNA. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively. The nucleotide sequence comprising the intended edit is integrated into the genomic DNA by HDR at the genomic target locus recognized (i.e., sufficiently complementary for hybridization) by the 5′ and 3′ homology arms.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks). A Cas9 endonuclease binds to and cleaves DNA at a site comprising a sequence complementary to its bound guide RNA (gRNA).

A Cas9 polynucleotide, nucleic acid, oligonucleotide, protein, polypeptide, or peptide refers to a molecule derived from any source. The molecule need not be physically derived from an organism, but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP_002989955, WP_038434062, WP_011528583); Campylobacter jejuni (WP_022552435, YP_002344900), Campylobacter coli (WP_060786116); Campylobacter fetus (WP_059434633); Corynebacterium ulcerans (NC_015683, NC_017317); Corynebacterium diphtheria (NC_016782, NC_016786); Enterococcus faecalis (WP_033919308); Spiroplasma syrphidicola (NC_021284); Prevotella intermedia (NC_017861); Spiroplasma taiwanense (NC_021846); Streptococcus iniae (NC_021314); Belliella baltica (NC_018010); Psychroflexus torquisl (NC_018721); Streptococcus thermophilus (YP_820832), Streptococcus mutans (WP_061046374, WP_024786433); Listeria innocua (NP_472073); Listeria monocytogenes (WP_061665472); Legionella pneumophila (WP_062726656); Staphylococcus aureus (WP_001573634); Francisella tularensis (WP_032729892, WP_014548420), Enterococcus faecalis (WP_033919308); Lactobacillus rhamnosus (WP_048482595, WP_032965177); and Neisseria meningitidis (WP_061704949, YP_002342100); all of which sequences (as entered by the date of filing of this application) are herein incorporated by reference. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein, wherein the variant retains biological activity, such as Cas9 site-directed endonuclease activity. See also Fonfara et al. (2014) Nucleic Acids Res. 42(4):2577-90; Kapitonov et al. (2015) J. Bacteriol. 198(5):797-807, Shmakov et al. (2015) Mol. Cell. 60(3):385-397, and Chylinski et al. (2014) Nucleic Acids Res. 42(10):6091-6105); for sequence comparisons and a discussion of genetic diversity and phylogenetic analysis of Cas9.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence, fusion proteins with heterologous and homologous leader sequences, with or without N-terminus methionine residues; immunologically tagged proteins; and the like.

### In Utero or Postnatal Treatment of Diseases Associated with Alternative Splicing

Methods and compositions for the delivery of therapeutic agents such as antisense oligonucleotides, small interfering RNAs (siRNAs), short hairpin RNAs (shRNAs), gene therapy agents, proteins, small molecules, CRISPR systems (gRNA/Cas9), and other such materials are provided for the in utero or postnatal treatment of diseases associated with alternative splicing. Compositions of the disclosure include nanoparticles for delivery of therapeutics, including those with nucleic acid scaffolding linked to a therapeutic agent that promote advantageous mRNA splicing phenotypes in cells when the compositions are delivered to a fetus in utero or after birth.

Such compositions may be used to treat diseases associated with alternative splicing. By “diseases associated with alternative splicing” is meant any disease associated with aberrant splicing of RNA. Alternative splicing may result, for example, in reduced levels of a protein, truncated protein variants, a reduction in functional protein, gene silencing variants, instability of a protein, disordered gene regulation, or variants that alter the function of a protein. Diseases associated with alternative splicing include, but are not limited to, spinal muscular atrophy, Angelman syndrome, myotonic dystrophy, Duchenne muscular dystrophy (DMD), choroideremia, Pompe disease, spinocerebellar ataxia, beta thalassemia, cancer, inflammatory conditions, Frasier syndrome, frontotemporal dementia and Parkinsonism linked to chromosome 17 (FTDP-17), atypical cystic fibrosis, diabetes, Usher syndrome, sickle cell anemia, thalassemias, Fanconi’s anemia, familial dysautonomia, Hutchinson-Gilford progeria syndrome (HGPS), hyperchole sterolemia, Prader-Willi syndrome, medium-chain acyl-CoA dehydrogenase (MCAD) deficiency, tauopathies, lysosomal storage disorders neurodevelopmental delay disorders, and metabolic disorders.

Nanoparticles used for delivery of therapeutic agents for treating diseases associated with alternative splicing have at least one dimension (e.g., a greatest dimension) in the range of from 1 nanometer (nm) to 1000 nm, from 20 nm to 750 nm, from 50 nm to 500 nm, including from 100 nm to 300 nm. The nanoparticle may have any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. In certain embodiments, the nanoparticle (e.g., a spherical or spheroid particle) has a greatest dimension of from 10 to 200 nm, e.g., from 30 to 100 nm. According to some embodiments, the greatest dimension of the nanoparticle (e.g., the diameter in the case of a spherical or spheroid nanoparticle) is greater than 10 nm but 500 nm or less, 450 nm or less, 400 nm or less, 350 nm or less, 300 nm or less, 250 nm or less, 200 nm or less, or 100 nm or less. In certain embodiments, the greatest dimension of the nanoparticle (e.g., the diameter in the case of a spherical or spheroid nanoparticle) is less than 500 nm, but 10 nm or greater, 20 nm or greater, 30 nm or greater, 40 nm or greater, 50 nm or greater, 60 nm or greater, 70 nm or greater, 80 nm or greater, 90 nm or greater, 100 nm or greater, 125 nm or greater, 150 nm or greater, 175 nm or greater, 200 nm or greater, 225 nm or greater, 250 nm or greater, 275 nm or greater, 300 nm or greater, 350 nm or greater, or 400 nm or greater.

The nanoparticle may be made of any suitable material or mixtures thereof. Suitable materials include, but are not limited to, organic or inorganic polymers, natural and synthetic polymers, including, but not limited to, agarose, cellulose, nitrocellulose, cellulose acetate, other cellulose derivatives, dextran, dextran-derivatives and dextran co-polymers, other polysaccharides, glass, silica gels, gelatin, polyvinyl pyrrolidone, rayon, nylon, polyethylene, polypropylene, polybutylene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamides, polyacrylamide blends, co-polymers of vinyl and acrylamide, methacrylates, methacrylate derivatives and co-polymers, polyethylene glycol, polylactic acid, poly(lactic-co-glycolic acid), polycaprolactone, other polymers and co-polymers with various functional groups, latex, butyl rubber and other synthetic rubbers, silicon, glass, insoluble protein, metals and metal oxides (e.g., gold, silver, nickel, platinum, iron oxide, titanium dioxide, and the like), metal alloys, metalloids, magnetic materials, and any combinations thereof. The nanoparticles may be magnetically responsive, e.g., by virtue of comprising one or more paramagnetic and/or superparamagnetic substances, such as for example, magnetite. Such paramagnetic and/or superparamagnetic substances may be embedded within a matrix of the nanoparticle, and/or may be disposed on an external and/or internal surface of the nanoparticle. Nanoparticles may also include liposomes, lipid nanoparticles, solid lipid nanoparticles, lipid-polymer hybrid nanoparticles, and the like. Such nanoparticles may comprise, without limitation, cationic lipids (e.g., 1,2-dioleoyl-sn-glycero-3-ethylphosphocholine, didodecyldimethylammonium bromide (DDAB), 3-dioleyloxy)propyl]-N,N,N-trimethylammonium chloride (DOTMA), 1,2-dioleyloxy-3-dimethylaminopropane (DODMA), 1, 2-oleinyl-3-trimethylammonium-propane (DOTAP), N-[1-(2,3-oleoyloxy)propyl]-N,N,N-trimethyl-ammonium methyl sulfate, dioleoylphosphatidyl ethanolamine (DOPE), oleic acid (OA), dimethyldioctadecyl ammonium bromide, N4-cholesteryl-spermine (GL67), and the like), phospholipids (e.g., phosphatidylcholine, phosphatidylethanolamine), sterols (e.g., cholesterol), PEGylated lipids, triglycerides (e.g. tristearin), diglycerides (e.g. glycerol bahenate), monoglycerides (e.g. glycerol monostearate), fatty acids (e.g. stearic acid), waxes (e.g. cetyl palmitate), sphingomyelins, bile salts (sodium taurocholate), or surfactants, and any combinations thereof.

Nanoparticles may be used to deliver small RNA particles such as siRNA or shRNA, including those that can be designed to alter gene expression, such as knocking down a repressor to increase expression of a target. Nanoparticle-based methods and compositions of the disclosure may also be used to deliver a protein, such as a missing protein not being naturally expressed by a subject or a CRISPR system to genetically modify a cell to eliminate a disease-causing mutation associated with alternative splicing.

Additionally, embodiments of the disclosure may be used to deliver newly designed therapeutics such as, for example, an antisense oligonucleotide that is made by sequencing nucleic acid from a subject and analyzing the resultant sequence data to identify a therapeutic target. The therapeutic (e.g., antisense oligonucleotide or shRNA) may then be designed to specifically target the newly identified therapeutic target. The designed therapeutic may be obtained (e.g., synthesized or ordered), packaged in nanoparticles, and delivered to the subject.

In some embodiments, an antisense oligonucleotide is used that inhibits unhealthy alternative splicing and promotes splicing of mRNA into preferred isoforms. The antisense oligonucleotides may be paired to a delivery mechanism that provides for delivery across the blood-brain barrier and/or longer half-life resulting in fewer doses that would be required for prior art intrathecal injections of antisense oligonucleotides. Such mechanisms may include carrier molecules such as nanoparticles, viruses, droplets such as liposomes, and pharmacological or physical methods to locally disrupt the blood-brain barrier. Methods and compositions of the disclosure may reduce or prevent the need for recurrent intrathecal injections and may also provide for the treatment of conditions such as SMN deficiency outside of the central nervous system.

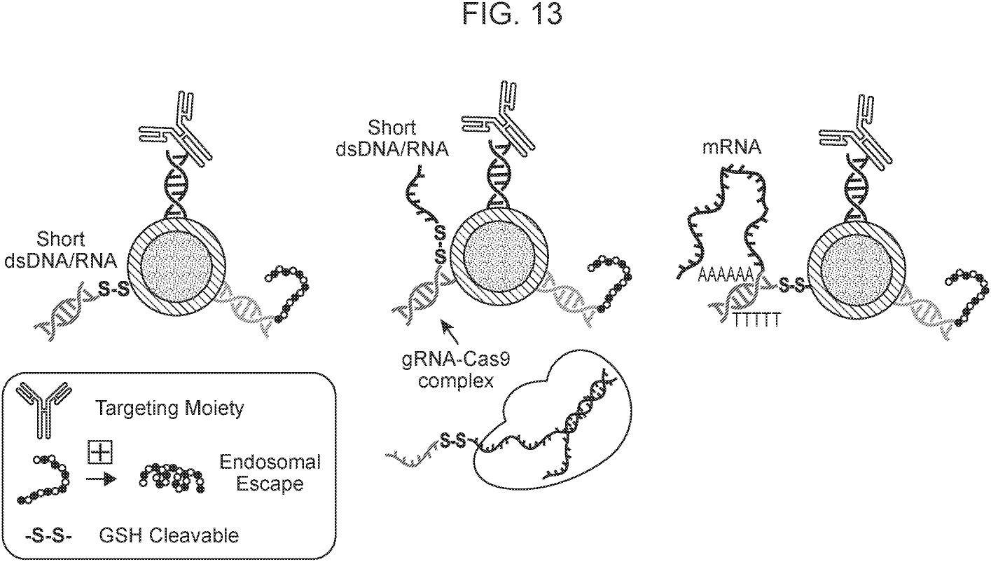

Certain embodiments make use of a nanoparticle coated with a nucleic acid scaffold (e.g., DNA or RNA). The nucleic acid scaffold provides useful features for loading therapeutic agents. FIG. 13 illustrates schematically how nanoparticles with nucleic acid scaffolds can be used to deliver various therapeutic agents including nucleic acids and CRISPR systems (e.g., gRNA and Cas9). For example, nucleic acids for gene therapy, antisense oligonucleotides, siRNAs, and shRNAs will bind to nucleic acids of the scaffold having complementary sequences. A Cas9-guide RNA complex will hybridize to nucleic acids of the scaffold having a sequence complementary to the guide RNA. In some embodiments, the scaffold comprises nucleic acids having a homopolymer chain, such as poly A, poly T, poly G, poly C, poly U, poly dA, poly dT, poly dG, poly dC, or poly dU, which can be used to bind therapeutic agents having a complementary homopolymer sequence. For example, mRNA having a poly A tail can be attached to a nucleic acid scaffold having a poly T or poly U sequence. Other therapeutic agents such as proteins and antibodies may be coupled to nucleic acids complementary to the scaffold nucleic acids to allow hybridization for attachment to the nanoparticle. Nanoparticles loaded with therapeutic agents may be safely injected into a fetus, where they are internalized into cells of a certain type. Such nanoparticles may be used to deliver therapeutic agents for treating diseases associated with alternative splicing.

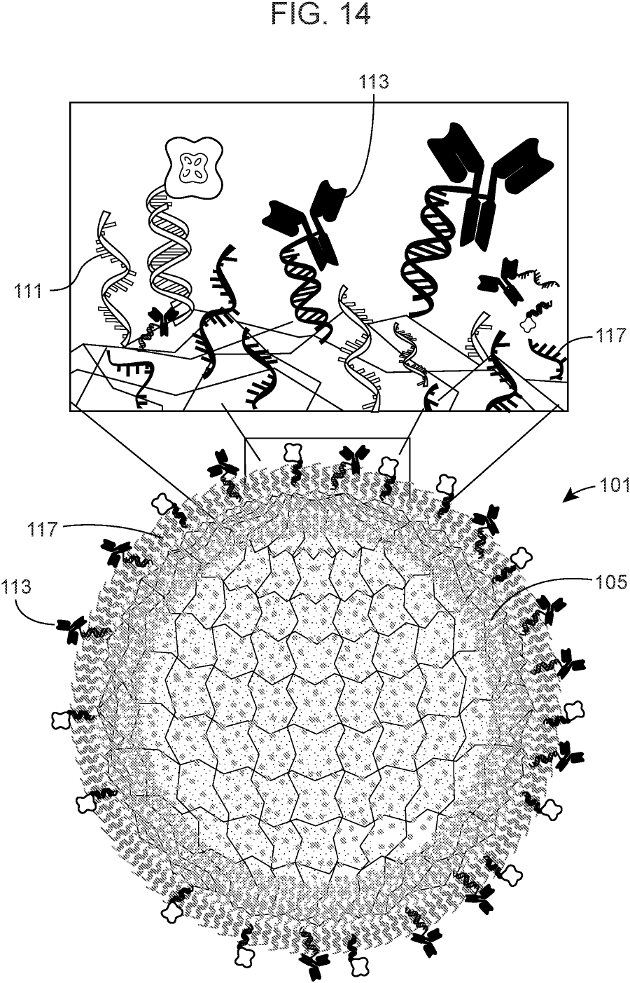

FIG. 14 shows an exemplary composition 101 for treating a disease. The composition 101 includes a nanoparticle 105 and a nucleic acid therapeutic such as an antisense oligonucleotide 111 carried by the nanoparticle. In some embodiments, the nanoparticle 105 is coated with antibodies 113 that can target delivery to particular cell types (e.g., motor neurons, hematopoietic stem cells, or CD45+ blood cells). This nanoparticle delivery platform is particularly amenable to the delivery of ASOs for the treatment of diseases involving alternative splicing, such as spinal muscular atrophy (SMA), Angelman syndrome, or muscular dystrophy, or the reduction of gene expression through gapmer-ASOs, for example, to decrease the expression of a gain-of-function missense mutation. Angelman syndrome can be treated, for example, using an ASO to activate the usually silenced paternal allele.

Developing cells in the fetus express unique antigens (or upregulate antigens that are not found at the same levels during adulthood), and coating the nanoparticle 205 with antibodies 113 designed to target fetal cell types may provide cell-type specificity for therapeutics, gene therapy, or gene editing. Table 2 lists antigens that may be used including, for example, certain antigens that are specific to developing cells in a fetus as well as certain antigens that may not be specific to a fetal cell, but may be accessible due to the unique milieu and migration patterns in the fetus.

In various embodiments, nanoparticles of the disclosure are provided for one of the disease applications listed in Table 2 and are targeted to a specific cell or tissue type, such as one of those listed in Table 2, by virtue of having a ligand for a specific target, such as one of the targets listed in Table 2. Suitable ligands include antibodies, antigen receptors, known cell-surface marker ligands, or any other ligand. In some embodiments, nanoparticles of the disclosure specifically target hematopoietic stem cells by using an antibody against the kit receptor. For example, nanoparticles may be coated with the 2B8 antibody, which reacts with CD117 (c-Kit), an -145 kDa type 1 transmembrane receptor for c-Kit ligand (stem cell factor/steel factor) that is broadly expressed on hematopoietic stem cells. Nanoparticles could also be targeted to other antigens such as CD34 and CD90 in humans, or coated with DLK-1 to target hepatocytes. Nanoparticles could also be targeted specifically to fetal muscle cells or satellite cells.

In the depicted embodiments, the nanoparticle 105 is coated with a nucleic acid scaffold 117. The scaffold 117 comprises DNA connected to or embedded in a surficial region of the nanoparticle 105. The DNA scaffold 117 provides a “handle” for linking biological molecules to the nanoparticle. For example, in some embodiments, the antisense oligonucleotides 111 are linked to DNA of the scaffold 117 (e.g., via disulfide bonds). In general, antisense oligonucleotides include small pieces of DNA or RNA that can bind to specific molecules of RNA. Such oligos may be used to block the ability of the RNA to make a protein or work in other ways. Antisense oligonucleotides may be used to block the production of proteins needed for cell growth. Antisense oligonucleotides generally include splice-switching oligonucleotides (SSOs). In general, splice-switching oligonucleotides (SSOs) are short, synthetic, antisense, optionally modified nucleic acids that base-pair with a pre-mRNA and disrupt the normal splicing repertoire of the transcript by blocking the RNA-RNA base-pairing or protein-RNA binding interactions that occur between components of the splicing machinery and the pre-mRNA. Splicing of pre-mRNA is required for the proper expression of the vast majority of protein-coding genes, and thus, targeting the process offers a means to manipulate protein production from a gene. Splicing modulation is particularly valuable in cases of disease caused by mutations that lead to disruption of normal splicing or when interfering with the normal splicing process of a gene transcript may be therapeutic. Compositions 101 of the disclosure may be used to deliver SSOs to treat disease associated with alternative splicing such as spinal muscle atrophy.

The depicted composition 101 is useful, for example, for delivering antisense oligonucleotides 111 that may be designed for manipulating exon-skipping in other diseases. For example, a mutation of GM-1 gangliosidosis maybe be treated with antisense oligonucleotides to manipulate alternative splicing. In preferred embodiments, the nanoparticles 105 are used for in utero delivery of antisense oligonucleotides to treat diseases associated with alternative splicing such as spinal muscular atrophy. This disclosed nanoparticles may include one or any combination of (i) antibody-mediated cell targeting/penetration, (ii) peptide-promoted endosomal escape, and (iii) cytosolic GSH-responsive ASO release. The nanoparticles may have a polymer inner regions comprising a polymer such as poly(lactic-co-glycolic acid) (PLGA). See Makadia, 2011, Poly Lactic-co-Glycolic Acid (PLGA) as biodegradable controlled drug delivery carrier, Polymers (Basel) 3:1377-1397, incorporated by reference. The inventors have developed and employed nanoparticles with a surface-embedded DNA scaffold. The particles generally have a diameter of between about 50 and 500 nm, with the ability to produce the nanoparticles 105 with diameters of about 100-200 nm, well suitable for intracellular delivery. In preferred embodiments, polymer-DNA (e.g., about 17 mer) amphiphilic conjugates are synthesized and incorporated during the emulsion protocol for particle fabrication, and the DNA scaffolds are driven to the surface through hydrophobic-hydrophilic interaction.

In other embodiments, the composition is used to deliver a nucleic acid therapeutic such as a short hairpin RNA (shRNA). For example, shRNA may be used to down-regulate gene expression for a gene of interest. The gene of interest may be repressor of an important gene. For example, the gene BCL11A represses fetal hemoglobin production. Knocking down BCL11A can lead to an increased hemoglobin production and is one promising approaching to treating sickle cell anemia. Where shRNA may knock down BCL11A, it may remain a challenge to deliver the therapeutic to target cells. A nanoparticle may be used to effectively deliver an shRNA and any related therapeutic tools such as helpful microRNAs or other features. The composition includes the nanoparticle 105 (e.g., a PLGA nanoparticle). The nanoparticle 105 preferably includes a scaffold 117 of “scaffold nucleic acid” (simply using the adjective scaffold to distinguish from the therapeutic nucleic acid that the nanoparticle delivers). The scaffold nucleic acid may be understood to be essentially embedded around an outermost shell of the nanoparticle, and the therapeutic nucleic acid may be attached to the nanoparticle via interactions with the scaffold nucleic acid, which interactions may include Watson-Crick base-pairing, disulfide bond formation, electrostatic interactions, others, or combinations thereof. In fact, the strength of interaction with, and thus the proclivity to release, the therapeutic nucleic acid from the nanoparticle can be “tuned” by engineering full or a limited amount of sequence complementarity between portions of the scaffold nucleic acid and the therapeutic nucleic acid. As mentioned, the therapeutic nucleic acid may be a shRNA, such as one of the shRNA discussed in Samakoglu, 2006, A genetic strategy to treat sickle cell anemia by coregulating globin transgene expression and RNA interference, Nat Biotech 24(1):89-94 and Guda, 2015, miRNA-embedded shRNAs for linage-specific BCL11A knockdown and hemoglobin F induction, Mol Ther 2399):1465-1474, incorporated by reference.

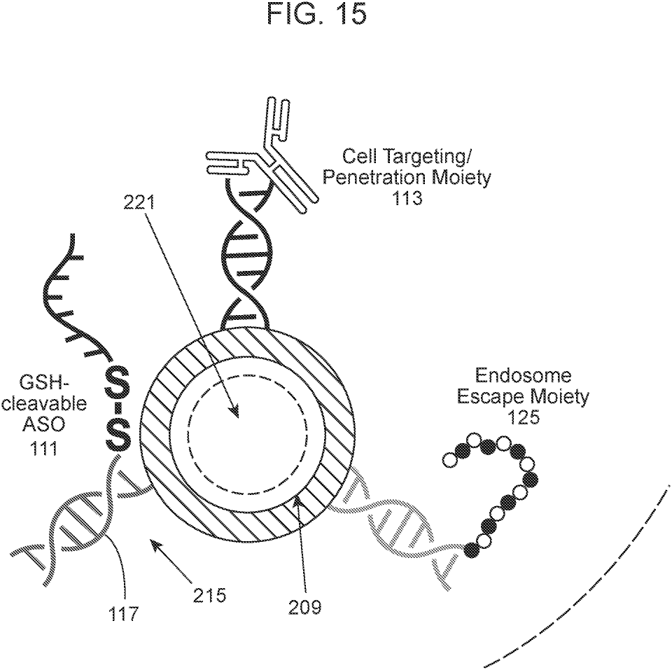

FIG. 15 shows features that may be included in a nanoparticle 105 of the disclosure. The nanoparticle 105 may have an inner region 209, which may comprise, e.g., a polymer (e.g., poly lactic-co-glycolic acid), metal (e.g., gold or silver), or liposome, and an outer region 215 that comprises a scaffold 117 of nucleic acids. In some embodiments, the therapeutic agent (e.g., antisense oligonucleotide) 111 is linked to the nucleic acids of the scaffold 117, e.g., via disulfide bonds. The nanoparticle 105 may comprise one or a plurality of endosomal escape peptides 125 linked to the nucleic acid of the scaffold 117. The nanoparticle 105 may have a plurality of targeting complexes 113 linked to the nucleic acid of the scaffold. The targeting complexes 113 may be antibodies that bind to cell-surface markers on fetal cells.

In some embodiments, the inner region 209 of the nanoparticle surrounds a core 221. The core 221 may contain a payload such as a small molecule, a protein, or a nucleic acid. For example, the core 221 may include gene editing reagents.

In preferred embodiments, the antisense oligonucleotide 111 is a splice-switching oligonucleotide (SSO) complementary to an mRNA from gene for which alternative splicing results in a disease. For example, the antisense oligonucleotide 111 may be complementary to a segment of a survival motor neuron (SMN) gene. In general, the particles 105 may be used to deliver antisense oligonucleotides 111 such as SSOs to ameliorate effects of a disease. Table 1 references certain SSOs that may be used by their genetic targets, the disease to be treated, and reference(s) reporting the SSO sequences.

TABLE 1

known genetic target of SSOs, disease to be treated, and reference(s) giving SSOs

| | | |
|---|---|---|
| Gene of target pre-mRNA | Disease | Reference, all incorporated by reference |
| SMN2 | Spinal muscle atrophy | US 8,946,183 B2 US 2014/0128449 A1 US 2014/0343127 A1 US 7,838,657 B2 US 2019/0030058 A1 US 9,885,040 B2 |
| Myostatin (growth differentiation factor 8) | Muscular atrophy | US 8,785,410 B2 |
| Dystrophin | Duchenne muscular dystrophy (DMD) | US 6,653,467 B1 US 8,232,384 B2 US 2014/0128449 A1 |
| Beta globin | Beta thalassemia | US 5,665,593 |
| Wilms tumor suppressor gene (WT1) | Cancer Frasier syndrome | US 2004/0043950 A1 |
| MAPT (protein tau) | Frontotemporal Dementia and Parkinsonism Linked to Chromosome 17 (FTDP-17) | US 2018/0161356 A1 |
| cystic fibrosis transmembrane conductance regulator (CFTR) | Atypical cystic fibrosis | US 2018/0155723 A1 |
| Soluble epidermal growth factor receptors 2 and 3 (HER2 and HER3) | Cancer | US 7,884,194 B2 |
| Tumor necrosis factor receptors (TNFR1 and TNFR2) | Inflammatory conditions | US 2019/0017052 A1 |
| androgen receptor (AR) | Cancer | US 2018/0334677 A1 |
| epithelial growth factor receptor (EGFR) | Cancer | US 2018/0334677 A1 |
| Insulin receptor (IR) | diabetes | |
| Ush1c | Usher syndrome | US 8,648,053 B2 |
| SCN2A | Neurodevelopmental delay | |
| Angelman Syndrome | Neurodevelopmental delay | |

In certain embodiments, the nanoparticles are linked to antisense oligonucleotides of about 10 to 35 bases in length, e.g., between about 12 to 25 nucleotides. The antisense oligonucleotides 111 preferably include a targeting base sequence that is complementary to a target region of a selected preprocessed mRNA coding for a selected protein, where the 5′ end of the target region is 1 to 25 bases downstream, preferably 2 to 20 bases downstream, and more preferably 2 to 15 bases downstream, of one splice acceptor site in the preprocessed mRNA, is effective to inhibit splicing at the one splice acceptor site and thus produce splice variant mRNA. Preferably, the antisense compound is one that does not activate RNase H. Such oligos may include morpholino oligomers, peptide nucleic acids, methylphosphonates, and 2′-O-alkyl or -allyl modified oligonucleotides, as known in the art. The antisense oligomers may be morpholino oligomers, which are composed of morpholino subunits of the form shown in U.S. Pat. Nos. 5,698,685, 5,217,866, 5,142,047, 5,034,506, 5,166,315, 5,521,063, and 5,506,337, all incorporated by reference. In a morpholino oligomer, (i) the morpholino groups are linked together by uncharged phosphorus-containing linkages, one to three atoms long, joining the morpholino nitrogen of one subunit to the 5′ exocyclic carbon of an adjacent subunit, and (ii) the base attached to the morpholino group is a purine or pyrimidine base-pairing moiety effective to bind, by base-specific hydrogen bonding, to a base in a polynucleotide. Preparation of such oligomers is described in detail in U.S. Pat. No. 5,185,444, incorporated by reference.

Such oligomers have shown high binding affinity for RNA targets, and the uncharged backbone favors uptake into cells and reduces non-specific binding interactions, relative to charged analogs such as phosphorothioates.

In preferred embodiments, the antisense oligonucleotide is complementary to, and hybridizes to, a messenger RNA (mRNA) when delivered to a fetal cell in utero. Preferably, the antisense oligonucleotide 111 is a splice-switching oligonucleotide (SSO). SSOs are oligos that hybridize to RNA transcripts and promote a favorable, or desired, splicing over a dis-favored or unhealthy splicing. In methods and compositions of the disclosure, SSOs are delivered to a fetus in utero, where—by endosomal uptake—the SSOs interact with fetal mRNA transcripts to promote a healthy phenotype.

SSOs, sometimes called splice-modulating drugs, may provide therapeutic benefit to some patients with conditions such as Duchenne muscular dystrophy (DMD) or spinal muscular atrophy (SMA). Existing SSOs include the SSO sold as EXONDYS 51 (eteplirsen) by Sarepta Therapeutics (Cambridge, MA), an antisense phosphorodiamidate morpholino oligomer (PMO) that targets splice enhancer motifs in the DMD pre-mRNA to exclude exon 51 and restore the dystrophin mRNA reading frame, disrupted by most deletions beginning at exon 52 or ending at exon 50. The composition may be used to deliver eteplirsen or another antisense oligonucleotide or SSO that targets splice enhancer motifs in the DMD pre-mRNA. The composition may include an antisense oligonucleotide targeting any suitable DMD exon. For example, the composition may be used to deliver an SSO for skipping of exon 44 (PRO044, BMN 044), exon 45 (SRP-4045, BMN 045), exon 51 (Eteplirsen, Kyndrisa) or exon 53 (SRP-4053, BMN 053). See Kryczka, 2014 Hum Gene Ther 25:587, incorporated by reference.

Another example includes the SSO sold as SPINRAZA (nusinersen) by Biogen (Cambridge, MA), a 2′- O-methoxyethyl antisense oligonucleotide (AO) on a phosphorothioate backbone that targets a splice silencer (ISS-N1) in survival motor neuron 2 (SMN2) intron 7 and promotes exon 7 selection and retention during pre-mRNA splicing. The composition may be used to deliver nusinersen or another antisense oligonucleotide or SSO that targets an SMN gene. Compositions 101 and methods of the disclosure may be used to address any disease known to be associated with alternative splicing. For example, some thalassemia is associated defect in splicing caused by a mutation in the human β-globin gene that creates a cryptic 5′ splice site, which is used preferentially over the natural site. See Dominski and Kole PNAS 90:8673, incorporated by reference. The composition may include an antisense oligonucleotide that hybridizes to, and masks the cryptic 5′ splice site associated with such incidences of thalassemia.

The SSOs may be used to increase SMN2 exon 7 splicing by targeting the 3′ splice site of exon 8 to result in an increase in the use of the 3′ splice site of exon 7 and, thereby, more exon 7 inclusion. Similar, SSOs may be used to increase exon 7 splicing by blocking putative splicing silencer elements surrounding exon 7. See Tisdale, 2015, J Neurosci 35:8691, incorporated by reference.

Compositions and methods of the disclosure may be used to deliver SSOs to address familial dysautonomia (FD), a rare inherited neurodegenerative disorder caused by a point mutation in the IKBKAP gene that results in defective splicing of its pre-mRNA. The point mutation in the IKBKAP gene weakens the 5′ splice site of exon 20, causing this exon to be skipped, thereby introducing a premature termination codon. Using a composition of the disclosure, an ASO may correct the splicing defect thus restoring normal expression levels of the full-length IKAP protein. The composition may be used to deliver ASOs targeting IKBKAP exon 20 or the adjoining intronic regions. See Sinha, 2018, Nucl Acids Res 10:4833, incorporated by reference.

Compositions and methods of the disclosure may be used to implement exon-skipping strategies to treat dysferlinopathies (for example, limb girdle muscular dystrophy type 2B) by delivering an antisense oligonucleotide that promotes the exclusion of exons 37 and/or 38 in a Dysf mutant. Compositions and methods of the disclosure may be used to target cryptic exons activated by deep intronic mutations causing choroideremia. See Garanto, 2018, Adv Exp Med Biol 1074:83, incorporated by reference. Compositions and methods of the disclosure may be used to deliver antisense oligonucleotides to treat Leber congenital amaurosis (see Garanto, 2016, Hum MOI Genet 25:2552, incorporated by reference); USH2A-associated retinal degeneration (see Sliijkerman, 2016, Mol Ther Nucl Ac 5:e381, incorporated by reference); to inhibit mis-splicing of harmonium in USH1C and exon inclusion to address a common splice variant causing adult-onset Pompe disease (see van der Wal, 2017, Mol Ther Nucl Ac 7:90, incorporated by reference); or to promote exon-skipping to treat spinocerebellar ataxia type 3, e.g., by skipping exon 10 to remove a pathogenic expanded polyglutamine repeat (see Toonen, 2017, Mol Ther Nucl Ac 8:232, incorporated by reference) or by skipping exons 8 and 9 to prevent proteolytic cleavage and generation of toxic protein fragments (see Toonen, 2016, Sci Rep 6:35200, incorporated by reference).

The nanoparticle may include a plurality of the cell targeting/penetration complexes (e.g., antibodies) that allow for specific cell type internalization. Any suitable targeting complexes may be used to decorate the nanoparticle. Preferably, the targeting complexes bind to cell-surface markers on the target cells of a certain cell type, tissue type, and/or developmental stage. For example, it may be most preferable to target fetal cells. The targeting complexes promote the endosomal uptake of the nanoparticles into the target cells. It may be preferable to have each nanoparticle decorated with a mixture of targeting complexes. For example, each nanoparticle may include a mixture of first targeting complexes that are specific to fetal cells and second targeting complexes specific to a cell or tissue type (e.g., neurons, stem cells, blood cells, etc.). Suitable targeting complexes include antibodies, aptamers, and proteins, proteoglycans, etc., with known cell-surface ligands to target. Table 2 lists certain cell surface markers/ targets.

TABLE 2

Targets and applications

| | | |
|---|---|---|
| Target | Cell/tissue type | Disease application (s) |
| C-kit receptor CD34 CD90 | Hematopoietic stem cells | Sickle cell Thalassemias Fanconi’s Anemia |
| DLK-1 | Hepatocytes | Lysosomal storage disorders (LSDs) |
| MCAM Pax7 CXCR4 VCAm1 ❏7-integrin CD34 | Myocytes, myoblasts, or satellite cells | Muscle atrophy DMD |
| mAb 2F7 Sox1 Pax6 Sox2 Nestin | Motor neurons | Spinal muscle atrophy |
| CD105, CD146 or CD141, Vimentin, VCAM, ICAM, VEGFR-1, VEGFR-2, VEGFR-3, ITGA5, ITGB5, CDH11 or CDH3 | Endothelial cells Fetal cells | Targeting fetal/ in utero stages |
| CK1, CK2, CK3, CK4, CK5, CK6, CK7, CK8, CK9, CK10, CK10, CK13, CK14, CK15, CK16, CK17, CK18 or CK19 | Epithelial cells Fetal cells | Targeting fetal/ in utero stages |
| KIF20A, EXO1, SKA3, NUF2, HJURP, CKAP2L, CDCA2, CDCA8, E2F7, BUB1 | Radial glia | Metabolic and neurodevelopmental disorders |
| BHLHE22, CAMKV, NEUROD2, GPR12, NEUROD6, MPPED1, RBFOX1, CLMP, DCX, PLS3, DLX2, PROX1, NYAP2, BMP3, PTPRR, ERBB4 | Developing neurons | Neurodevelopmental disorders |
| TREM2, FGD2, RGS1, CLEC7A, C1QB, FCER1G, GPR183, ITGAX, P2RY13, C1QC, Iba 1, CX3CR1 | Microglia | Metabolic and neurodevelopmental disorders |
| B3GNT7, PCDH15, LHFPL3, OLIG2, LUZP2, TNR, BCAN, OLIG1, XYLT1 | Oligodendrocyte precursor cells | Metabolic and neurodevelopmental disorders |

The nanoparticle may further comprise the targeting complexes, endosomal escape peptide (e.g. GALA) that refold to helical structures to promote the release of nanocarriers from endosomes, and the antisense oligonucleotides linked to the scaffold-complementary strand through a disulfide bond, which is cleavable as exposed to cytosolic glutathione (GSH) and the core may optionally be loaded with additional endosomal escape reagents (e.g. quinacrine). In preferred embodiments, the targeting complexes are ligands for natal cell-surface markers such as cell-type targeting antibodies.

The disclosure provides methods of treating a patient in utero for a disease associated with alternative splicing.

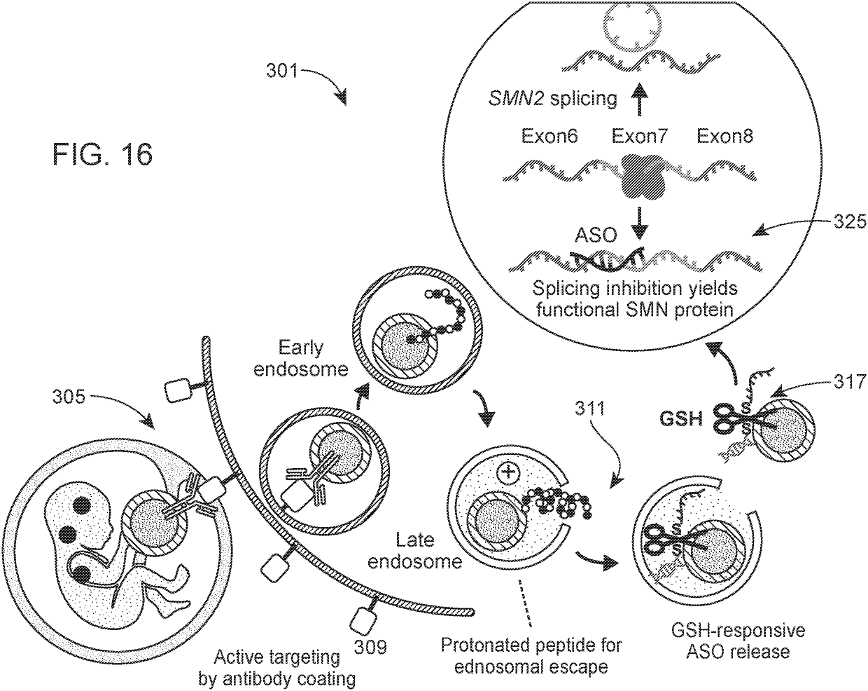

FIG. 16 shows progress through a method 301 of treating a patient in utero for a disease associated with alternative splicing. The method 301 may include injecting 305 a composition 101 comprising a nanoparticle 105 into circulation in a fetus. In SMA embodiments, the nanoparticle 105 is coated with targeting complexes that target the nanoparticles to neurons or precursors thereof. After injecting 305 the nanoparticle into circulation in a fetus, the method 301 may include targeting 309 the nanoparticle to cells of specific type by means of the targeting complexes 113; releasing 311 the nanoparticles 105 into cytoplasm by means of the escape peptides 125; and releasing 317 the antisense oligonucleotides 111 into the cytoplasm by means of the S—S bonds that are cleaved upon exposure to glutathione in the cytosol.

The released antisense oligonucleotide 111 (ASO) can then bind 325 to an mRNA to prevent splicing of the mRNA into a disease-associated isoform in the fetus. In the methods, the injecting 305 and binding 325 steps are essential. The targeting 309, particle releasing 311, and oligo releasing 317 steps may each separately or in any combination may be included in various preferred embodiments. In the method 301, the nanoparticle 105 preferably includes endosomal escape peptides 125 that cause release of the nanoparticles into cytosol of the neurons or the precursors thereof. Preferably, the nanoparticle 105 is linked to antisense oligonucleotides 111 by disulfide bonds that are cleaved upon exposure to glutathione in the cytosol to release the oligos to allow them to function as SSOs. In the SMA embodiments, the SSO binds 325 to an SMN mRNA and prevents formation of an isoform associated with spinal muscle atrophy. Thus, the antisense oligonucleotide 111 is preferably an SSO that is complementary to, and hybridizes to, an mRNA from a gene selected from the group consisting of: survival motor neuron 1, survival motor neuron 2, β-globin, the IKBKAP gene, or the DMD gene (SMN2 in SMA embodiments). Preferably, an inner region 209 of the nanoparticle 105 comprises PLGA and an outer region 215 of the nanoparticle 105 comprises a scaffold 117 of nucleic acid linked to the antisense oligonucleotides 111, targeting complexes 113, and endosomal escape peptides 125. The antisense oligonucleotide 111 is a splice-switching oligonucleotide (SSO) complementary to an mRNA from a gene associated with a disease.

In the compositions and the methods, the antisense oligonucleotides are preferably between about 10 and 35 nucleotides in length. Additionally, one or more nucleotides in the antisense oligonucleotide includes a modification to prevent degradation. The modifications may include base methylation; phosphorothiate (PS) backbone modification; 2′-O-methyl (2′-OMe); 2′-O-methoxyethyl (2′-MOE); Locked nucleic acid (LNA); or phosphorodiamidate morpholinos (PMOs).

For the intracellular delivery of ASOs, the disclosure provides nanocarriers that may include three functional moieties on surface: (i) cell targeting/penetration antibodies that allow for specific cell type internalization; (ii) endosomal escape peptide (e.g. GALA) that refolds to helical structures to promote the release of nanocarriers from endosomes, and (iii) therapeutic ASOs linked to the scaffold-complementary strand through a disulfide bond, which is cleavable as exposed to cytosolic glutathione (GSH). In this design, the coverage of targeting antibodies and endosomal escape peptide can provide a steric protection for embedded ASO from enzymatic degradation before cellular uptake. Meanwhile, the degradability of nanocarriers can be tuned through using different polymers, and the core can be loaded with additional endosomal escape reagents (e.g. quinacrine) through double-emulsion method if necessary.

Other embodiments and techniques are within the scope of the disclosure. For example, embodiments of the disclosure relate to gene therapy and gene editing, in utero, to treat diseases that are associated with alternative splicing. For example, in gene therapy embodiments, a composition may be used to deliver a gene to a fetus. In some spinal muscle atrophy embodiments, the DNA-coated nanoparticle 105 is also be used to deliver a plasmid carrying the wild-type SMN1 gene. In gene editing embodiments, gene editing reagents (such as a Cas endonuclease or nucleic acid encoding the Cas endonuclease and at least one guide RNA) may be delivered. Gene editing (CRISPR-Cas9, TALENs, ZFNs) may be used to edit the gene SMN1 to correct or ameliorate the impact of the disease-causing variant or variants, increasing the quantity of functional SMN protein. Such a strategy may be effective in type 0 SMA, which is often fatal in utero and occurs in the presence of few copies of SMN2, so may be less amenable to approaches that focus on modification of SMN2. Due to the early onset of type 0 SMA, the treatment is given in utero. Gene therapy/ gene editing embodiments involve the in utero delivery (via injection into a fetal liver or heart or into circulation by injection into an umbilical cord) of a gene copy of gene editing reagents suing a viral vector (e.g. AAV9) or a non-viral delivery vector such as a nanoparticle 105. Such approaches are effective with a systemic injection of these reagents (via the umbilical vein or an intracardiac or hepatic injection), since the blood-brain barrier is less developed and more permeable in the fetus. Due to the relatively accessible fetal blood-brain barrier, intrathecal injections are not required for treating a fetus with gene therapy or gene editing reagents, and by systemic injection, those materials will likely be effective in reaching their targets (e.g., delivery of gene editing reagents for successfully editing motor neurons). Such a strategy minimizes or prevents the need for recurrent intrathecal injections, maintains the gene regulatory environment of a gene such as SMN1, and may treat the consequences of SMN deficiency inside and outside the central nervous system. The specific therapy can depend on the exact nature of the SMN1 variant in affected individuals and would be ineffective in cases with homozygous deletions of SMN1.

In gene therapy embodiments for SMA, a DNA-coated nanoparticle is used to deliver a plasmid carrying the wild-type SMN1 gene (the nanoparticle 105 may be preferable to prior art aav9 vectors due to the biocompatibility of the PLGA and the targeting offered by the targeting complexes). In gene editing embodiments, gene editing reagents are delivered to edit, for example, the gene SMN2 to increase the quantity of functional SMN protein. In some embodiments, Cas endonuclease (or a nucleic acid encoding the Cas endonuclease) are delivered with a segment of replacement DNA that will be “swapped into” the SMN2 gene, after cleavage of that gene by the Cas endonuclease, by homology-directed repair. This approach may be used for editing the nucleotide that differs in between SMN1 and SMN2 in exon 7 to promote inclusion of exon 7 in the SMN protein product of SMN2.

The reagents for editing would be delivered using either viral vectors (e.g., AAV9) or non-viral methods (e.g., nanoparticles). Of note, nanoparticles could be targeted to motor neurons using complexes (e.g., antibodies) that allow localization of the nanoparticles to a cell type of interest. This strategy would reduce or prevent the need for recurrent intrathecal injections, maintain the gene regulatory environment of SMN2, and treat the consequences of SMN deficiency inside and outside the central nervous system. The resulting therapy would potentially be applicable in all cases of SMA, though the dosage by need to differ based on SMN2 copy number. The disclosure includes compositions for the gene editing embodiments.

The disclosure provides a composition that includes gene-editing reagents, or nucleic acid encoding the gene-editing reagents, wherein the gene-editing reagents are targeted to a gene for which a variant promotes an alternative splicing of mRNA that causes a disease. When the composition is delivered to a fetus in utero, the gene editing reagents correct or ameliorate the effect of the variant to thereby inhibit the alternative splicing. The gene editing reagents (or nucleic acid encoding them) are preferably packaged using a nanoparticle for delivery. In some embodiments, nucleic acid (e.g., Cas9 plasmid) is used. In some embodiments, an inner region of the nanoparticle comprises a polymer (e.g., PLGA), a metal, or a liposome, and an outer region of the nanoparticle comprises a scaffold of nucleic acid. The scaffold may be decorated with targeting complexes and/or endosomal escape peptides. The gene editing reagents may include zinc-finger nucleases (ZFNs), transcription activator-like effector nucleases (TALENs), peptide-nucleic acids (PNAs) or a Cas endonuclease. In certain embodiments, the gene editing reagents include a Cas endonuclease complexed with a guide RNA as a ribonucleoprotein (RNP) within the nanoparticle (e.g., with the RNP packed into the core 221 of the nanoparticle 105). The guide RNA targets the Cas endonuclease to a survival motor neuron gene to inhibit alternative splicing of the SMN gene to inhibit spinal muscle atrophy in the fetus.

Other embodiments described herein will be apparent to those skilled in the art from consideration of the specification and practice of the invention disclosed herein. All U.S. Patents and other references noted herein for whatever reason are specifically incorporated by reference. The specification and examples should be considered exemplary only with the true scope and spirit of the invention indicated by the following claims.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-39 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A composition for treating a genetic condition, the composition comprising:
a nanoparticle; and
an antisense oligonucleotide carried by the nanoparticle,

2. The composition of aspect 1, wherein the antisense oligonucleotide is complementary to, and hybridizes to, a messenger RNA (mRNA) when delivered to a fetal cell.

3. The composition of aspect 2, wherein the mRNA is transcribed from a survival motor neuron gene.

4. The composition of any one of aspects 1-3, wherein the antisense oligonucleotide is a splice-switching oligonucleotide (SSO).

5. The composition of any one of aspects 1-4, wherein the nanoparticle further comprises a plurality of targeting complexes.

6. The composition of aspect 4 or 5, wherein the antisense oligonucleotide is an SSO that is complementary to, and hybridizes to, an mRNA from a gene selected from the group consisting of survival motor neuron 1, survival motor neuron 2, β-globin, the IKBKAP gene, UBE3a, genes for other developmental disorders, and the DMD gene.

7. The composition of any one of aspects 1-6, wherein the nanoparticle comprises:
an inner region comprising a polymer, a metal, or a liposome; and
an outer region comprising a scaffold of nucleic acid.

8. The composition of aspect 7, wherein the antisense oligonucleotide is linked to the nucleic acid of the scaffold.

9. The composition of aspect 7 or 8, further comprising one or a plurality of endosomal escape peptides linked to the nucleic acid of the scaffold.

10. The composition of any one of aspects 7-9, further comprising a plurality of targeting complexes linked to the nucleic acid of the scaffold.

11. The composition of aspect 10, wherein the targeting complexes comprise antibodies that bind to cell-surface markers on fetal cells or cells after birth.

12. The composition of any one of aspects 7-11, wherein the antisense oligonucleotide is linked to the nucleic acid of the scaffold by disulfide bonds.

13. The composition of aspect 12, further comprising targeting antibodies and/or escape peptides inked to the nucleic acid of the scaffold.

14. The composition of any one of aspects 7-13, wherein the polymer comprises poly lactic-co-glycolic acid (PLGA).

15. The composition of any one of aspects 7-14, further wherein the inner region of the nanoparticle surrounds a core that contains a payload.

16. The composition of aspect 15, wherein the payload comprises one or more of a small molecule, a protein, and a nucleic acid.

17. The composition of any one of aspects 12-16, wherein the antisense oligonucleotide is a splice-switching oligonucleotide (SSO) complementary to an mRNA from a survival motor neuron (SMN) gene.

18. The composition of aspect 17, wherein, when the nanoparticle is injected into circulation in a fetus:
the targeting complexes target the nanoparticles to neurons or precursors thereof;
the endosomal escape peptides cause release of the nanoparticles into cytosol of the neurons or the precursors thereof;
the SSO is released from the nanoparticle upon exposure to glutathione in the cytosol; and
the SSO binds to an SMN mRNA and prevents formation of an isoform associated with spinal muscle atrophy.

19. The composition of aspect 1, wherein an inner region of the nanoparticle comprises a PLGA and an outer region of the nanoparticle comprises a scaffold of nucleic acid linked to the antisense oligonucleotides, targeting complexes, and endosomal escape peptides, wherein the antisense oligonucleotide is a splice-switching oligonucleotide (SSO) complementary to an mRNA from a gene associated with a disease, wherein, when the nanoparticle is injected into circulation in a fetus:
the targeting complexes target the nanoparticles to cells of a specific type;
the escape peptides cause release of the nanoparticles into cytosol of the cells;
the SSO is released from the nanoparticle into the cytosol; and
the SSO binds to an mRNA and prevents formation of splicing of the mRNA into a disease-associated isoform.

20. The composition of aspect 1, wherein:
the antisense oligonucleotide is between about 10 and 35 nucleotides in length; or
one or more nucleotides in the antisense oligonucleotide includes a modification to prevent degradation by RNase, the modification selected from the group consisting of: base methylation; phosphorothiate (PS) backbone modification; 2′-O-methyl (2′-OMe); 2′-O-methoxyethyl (2′-MOE); Locked nucleic acid (LNA); and phosphorodiamidate morpholinos (PMOs).

21. The composition of aspect 1, wherein the gene-editing reagent is targeted to a gene for which a variant promotes an alternative splicing of mRNA that causes a disease, such that when the composition is delivered to a fetus in utero, the gene editing reagent corrects or ameliorates the effect of the variant to thereby inhibit the alternative splicing.

22. The composition of aspect 21, wherein the gene-editing reagents, or the nucleic acid encoding the gene-editing reagents, are packaged in a nanoparticle for delivery, wherein an inner region of the nanoparticle comprises a polymer, metal, or liposome, and an outer region of the nanoparticle comprises a scaffold of scaffold nucleic acid.

23. The composition of aspect 22, further comprising a plurality of targeting complexes linked to the nanoparticle.

24. The composition of aspect 22 or 23, further comprising endosomal escape peptides linked to the nanoparticle.

25. The composition of any one of aspects 22-24, wherein the polymer comprises PLGA.

26. The composition of any one of aspects 22-25, wherein the gene editing reagents include a zinc-finger nuclease (ZFN), a transcription activator-like effector nuclease (TALEN), or a peptide-nucleic acid (PNA).

27. The composition of any one of aspects 22-25, wherein the nanoparticle includes the nucleic acids encoding the gene editing reagents and the gene editing reagents include a Cas endonuclease.

28. The composition of any one of aspects 22-27, wherein the gene editing reagents include a Cas endonuclease complexed with a guide RNA as a ribonucleoprotein (RNP) within the nanoparticle.

29. The composition of aspect 28, wherein the guide RNA targets the Cas endonuclease to a gene to inhibit formation of a splicing isoform of a transcript of the gene.

30. The composition of aspect 28, wherein the guide RNA targets the Cas endonuclease to a survival motor neuron gene to inhibit alternative splicing of the SMN gene to inhibit spinal muscle atrophy in the fetus.

31. A therapeutic composition comprising:
a nanoparticle; and
a therapeutic agent carried by or on the nanoparticle.

32. The composition of aspect 31, wherein the therapeutic agent comprises a combination of small molecules, nucleotide sequences, and/or proteins.

33. The composition of aspect 31 or 32, wherein the therapeutic agent comprises one or more nucleotide sequences.

34. The composition of any one of aspects 31-33, wherein the therapeutic agent comprises one or more antisense oligonucleotides (ASO).

35. The composition of aspect 34, wherein: the antisense oligonucleotides are between about 10 and 35 nucleotides in length; and one or more nucleotides in the antisense oligonucleotide includes one or more modifications to prevent degradation, improve RNA binding efficiency, and/or reduce toxicity, the modification selected from the group including: base methylation; phosphorothiate (PS) backbone modification; 2′-O-methyl (2′-OMe); 2′-O-methoxyethyl (2′-MOE); locked nucleic acid (LNA); and phosphorodiamidate morpholinos (PMOs).

36. The composition of any one of aspects 31-35, wherein the therapeutic agent comprises one or more splice-switching oligonucleotides (SSO).

37. The composition of any one of aspects 31-36, wherein the therapeutic agent comprises one or more short hairpin RNAs (shRNA).

38. The composition of any one of aspects 31-37, wherein the therapeutic agent comprises one or more small interfering RNAs (siRNA).

39. The composition of any one of aspects 31-38, wherein the therapeutic agent is designed to induce immune tolerance when delivered in utero or after birth.

40. The composition of any one of aspects 31-39, wherein the therapeutic agent comprises one or more nucleotide sequences complementary to DNA.

41. The composition of any one of aspects 31-40, wherein the therapeutic agent comprises one or more nucleotide sequences complementary to RNA.

42. The composition of any one of aspect 31-41, wherein the therapeutic agent comprises one or more nucleotide sequences complementary to the RNA of a gene selected from the group consisting of survival motor neuron 1, survival motor neuron 2, P-globin, blc11a, the IKBKAP gene, the DMD gene, the UBE3A gene, the UBE3A-ATS gene, the SCN2A gene, the SCN8A gene, the SCN3A gene, and genes for other developmental disorders.

43. The composition of any one of aspect 31-42, wherein the therapeutic agent comprises one or more nucleotide sequences complementary to the RNA of a gene with multiple isoforms occurring physiologically with the objective of increasing the proportion of transcripts containing specific exons.

44. The composition of any one of aspects 31-43, wherein the therapeutic agent comprises one or more nucleotide sequences complementary to the RNA of a gene in which a genetic variant disrupts physiological splicing with the objective of restoring normal splicing behavior.

45. The composition of any one of aspects 31-44, wherein the therapeutic agent comprises one or more nucleotide sequences complementary to the RNA of a gene in which a genetic variant leads to an encoded protein that has a gain-of-function or dominant negative effect with the objective of decreasing the quantity of the abnormal RNA or encoded protein.

46. The composition of any one of aspects 31-45, wherein the therapeutic agent comprises one or more nucleotide sequences complementary to the genome of a pathogen.

47. The composition of any one of aspects 31-46, wherein the nanoparticle further comprises a plurality of targeting complexes.

48. The composition of any one of aspects 31-47, further comprising one or a plurality of endosomal escape peptides linked to the nucleic acid of the scaffold.

49. The composition of any one of aspects 31-48, further comprising reagents to facilitate passage across the blood-brain barrier.

50. The composition of any one of aspects 31-49, wherein the nanoparticle comprises: an inner region comprising a polymer, a metal, or a liposome; and an outer region comprising a scaffold of nucleic acid.

51. The composition of aspect 50, further comprising a plurality of targeting complexes linked to the nucleic acid of the scaffold.

52. The composition of aspect 51, wherein the targeting complexes comprise antibodies that bind to cell-surface markers on fetal cells or cells after birth.

53. The composition of any one of aspects 50-52, wherein the polymer comprises poly lactic-co-glycolic acid (PLGA).

54. The composition of any one of aspects 50-53, further wherein the inner region of the nanoparticle surrounds a core that contains a payload.

55. The composition of aspect 54, wherein the payload comprises one or more of a small molecule, a protein, and a nucleotide sequence.

56. The composition of aspect 54 or 55, wherein one or more nucleotide sequences are carried within the polymer or core of the nanoparticle.

57. The composition of any one of aspects 50-56, wherein one or more nucleotide sequences are linked to the nucleic acid of the scaffold.

58. The composition of aspect 57, wherein the nucleotide sequences are splice-switching oligonucleotides (SSOs) complementary to RNA from a survival motor neuron (SMN) gene.

59. The composition of aspect 57, wherein the nucleotide sequences are splice-switching oligonucleotides (SSOs) complementary to RNA from the DMD gene.

60. The composition of aspect 57, wherein the nucleotide sequences are antisense oligonucleotides (ASOs) complementary to an antisense RNA or IncRNA including the UBE3A-ATS gene or XIST gene.

61. The composition of any one of aspects 50-60, wherein one or more nucleotide sequences are linked to the nucleic acid of the scaffold by disulfide bonds.

62. The composition of any one of aspects 50-61, comprising a combination of nucleotide sequences and proteins, including targeting antibodies and/or escape peptides, linked to the nucleic acid of the scaffold.

63. The composition of any one of aspects 50-62, comprising a combination of small molecules, nucleotide sequences and proteins, including targeting antibodies and/or escape peptides, linked to the nucleic acid of the scaffold or contained within the polymer or core of the nanoparticle.

64. The composition of any one of aspects 50-63, wherein the scaffold of nucleic acid contains multiple nucleotide sequences of varying length and varying degrees of complementarity to a therapeutic nucleotide sequence.

65. The composition of any one of aspects 50-64, wherein, when the nanoparticle is injected into circulation before or after birth:
the targeting complexes target the nanoparticles to neurons or precursors thereof;
the endosomal escape peptides cause release of the nanoparticles into cytosol of the neurons or the precursors thereof;
the nucleotide sequences are released from the nanoparticle upon exposure to glutathione in the cytosol; and
the nucleotide sequences bind to RNA to modify splicing or degrade the RNA.

66. The composition of aspect 50, wherein an inner region of the nanoparticle comprises a PLGA and an outer region of the nanoparticle comprises a scaffold of nucleic acid linked to nucleotide sequences, targeting complexes, and endosomal escape peptides, wherein the nucleotide sequences are complementary to an RNA from a gene associated with a disease, wherein, when the nanoparticle is injected into circulation in a fetus: the targeting complexes target the nanoparticles to cells of a specific type; the escape peptides cause release of the nanoparticles into cytosol of the cells; the nucleotide sequences are released from the nanoparticle into the cytosol; and the nucleotide sequences bind to RNA to modify splicing or degrade the RNA.

67. The composition of any one of aspects 31-66, wherein the therapeutic agent includes splice-switching oligonucleotides (SSO) complementary to an SMN gene RNA and induce the generation of isoforms that produce stable and functional protein to treat spinal muscular atrophy.

68. The composition of any one of aspects 31-67, wherein the therapeutic agent includes splice-switching oligonucleotides (SSO) complementary to genes in which a stop codon leads to degradation of the RNA of one or more isoforms with the intent to increase expression.

69. The composition of any one of aspects 57-68, in which the nucleotide sequences have a sequence that targets a specific gene and in which: the gene to be targeted is DMD to skip exons with genetic variants leading to muscular dystrophy; or the gene to be targeted is SCN1A to skip exons that would lead to nonsense-mediated decay as a treatment for Dravet syndrome.

70. The composition of any one of aspects 57-68, wherein the nucleotide sequences are antisense oligonucleotides (ASOs) complementary to the UBE3A-ATS RNA leading to its degradation resulting in upregulation of the UBE3A gene to treat Angelman syndrome.

71. A composition comprising:
gene-editing reagents, or a nucleic acid comprising nucleotide sequences encoding the gene-editing reagents, wherein the gene-editing reagents are targeted to a gene for which a variant contributes to a disease, such that when the composition is delivered to a fetus in utero, the gene editing reagents correct or ameliorate the effect of the variant; or
gene-editing reagents, or nucleotide sequences encoding the gene-editing reagents, wherein the gene-editing reagents are targeted to a gene that modifies a disease process, such that when the composition is delivered to a fetus in utero, the gene editing reagents correct or ameliorate the disease process; or
reagents targeted to the cis regulatory regions of a gene, or nucleotide sequences encoding the reagents, wherein the reagents are targeted to a gene for which a variant causes a disease process, such that when the composition is delivered to a fetus in utero, the reagents correct or ameliorate the disease process by increasing gene expression, decreasing gene expression or modifying splicing and isoform usage.

72. The composition of aspect 71, wherein the reagents, or the nucleotide sequences encoding the reagents, are packaged with a nanoparticle for delivery.

73. The composition of aspect 72, wherein the nanoparticle comprises:
an inner region comprising a polymer, a metal, or a liposome; and
an outer region comprising a scaffold of nucleic acid.

74. The composition of aspect 73, wherein the polymer comprises PLGA.

75. The composition of any one of aspects 72-74, further comprising a plurality of targeting complexes linked to the nanoparticle.

76. The composition of any one of aspects 72-75, further comprising endosomal escape peptides linked to the nanoparticle.

77. The composition of any one of aspects 72-76, wherein the gene editing reagents include a zinc-finger nuclease (ZFN), a transcription activator-like effector nuclease (TALEN), a peptide-nucleic acid (PNA), or a Cas endonuclease.

78. The composition of any one of aspects 72-77, wherein the nanoparticle includes nucleotide sequences encoding the gene editing reagents and the gene editing reagents include a Cas endonuclease.

79. The composition of any one of aspects 72-78, wherein the gene editing reagents include a Cas endonuclease complexed with a guide RNA as a ribonucleoprotein (RNP) within the nanoparticle.

80. The composition of aspect 79, wherein the guide RNA targets the Cas endonuclease to a survival motor neuron gene to modify splicing of the SMN gene to produce stable and functional SMN protein to treat spinal muscle atrophy in the fetus.

81. The composition of aspect 79, wherein the guide RNA targets the Cas endonuclease to the CFTR gene to produce stable and functional CFTR protein to treat cystic fibrosis in the fetus.

82. The composition of any one of aspects 72-81, wherein the nanoparticle includes nucleotide sequences encoding a gene to replace one or more copies that are defective leading to disease in an individual.

83. The composition of aspect 82, wherein the gene to be replaced is: CFTR, HBB, SERPINA1, SLC26A4, KCNJ10, GALNS, DMD, F8, F9, F9, HBA2, HBA1, FMR1, HGSNAT, SFTPB, SGSH, SMN1, GBA, or SCARB2.

84. A composition comprising: a nanoparticle; a payload carried by the nanoparticle; and one or more targeting complexes linked to the nanoparticle.

85. The composition of aspect 84, wherein the nanoparticle comprises: an inner region comprising a polymer, a metal, or a liposome; and an outer region comprising a scaffold of nucleic acid.

86. The composition of aspect 85, wherein the payload is linked to the nucleic acid of the scaffold.

87. The composition of aspect 85 or 86, further comprising one or more endosomal escape peptides linked to the nucleic acid of the scaffold.

88. The composition of any one of aspects 84-87, wherein the targeting complexes comprise antibodies that bind to cell-surface markers on stem cells.

89. The composition of aspect 88, wherein the antibodies comprise α-c-kit antibodies.

90. The composition of any one of aspects 84-89, wherein the nanoparticle has a coating comprising antibodies targeting c-kit⁺ cells.

91. The composition of any one of aspects 85-90, wherein the polymer comprises poly lactic-co-glycolic acid (PLGA).

92. The composition of any one of aspects 85-91, wherein the inner region of the nanoparticle surrounds a core that contains a payload.

93. The composition of any one of aspects 84-92, wherein the payload comprises one or more of a small molecule, a protein, and a nucleic acid.

94. The composition of any one of aspects 84-93, wherein the payload comprises gene editing reagents or nucleic acids encoding the gene editing reagents.

95. The composition of aspect 94, wherein the gene editing reagents include at least one cas9 endonuclease and a guide RNA.

96. The composition of aspect 95, wherein the payload includes a mRNA, a plasmid, or a viral vector encoding at least one cas9 endonuclease and/or a guide RNA.

97. A method comprising delivering a composition according to any one of aspects of 84-96 to stem cells to introduce the payload into the stem cells.

98. The method of aspect 97, wherein the stem cells are hematopoietic stem cells (HSCs).

99. The method of aspect 97 or 98, wherein the delivery is performed in vitro or in vivo.

100. The method of any one of aspects 97-99, wherein the nanoparticle comprises:
an inner region comprising a polymer;
an outer region comprising a scaffold of nucleic acid; and
a coating of anti C-kit antibodies.
101. The method of aspect 100, wherein the polymer is poly lactic-co-glycolic acid (PLGA).

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Targeting Hematopoietic Stem Cells Using Antibodies Against the C-Kit Receptor

The C-kit receptor is one of the most important markers for identifying hematopoietic stem cells and their differentiation lineages. It is used here as a binding target for HSCs and the inducer for cell uptake of nanocarriers. Anti-c-kit antibody (clone 2B8) was conjugated to nanoparticles using streptavidin-biotin based chemistry for c-kit receptor targeting. Fetal liver cells were harvested and in vitro cultured with the supplementation of anti-c-kit-coated nanoparticles at different doses. We observed specific association of anti-C-kit-coated nanoparticles with C-kit positive cells after 1 hour and 5 hours’ co-incubation, compared to nanoparticles coated with isotype control antibody.

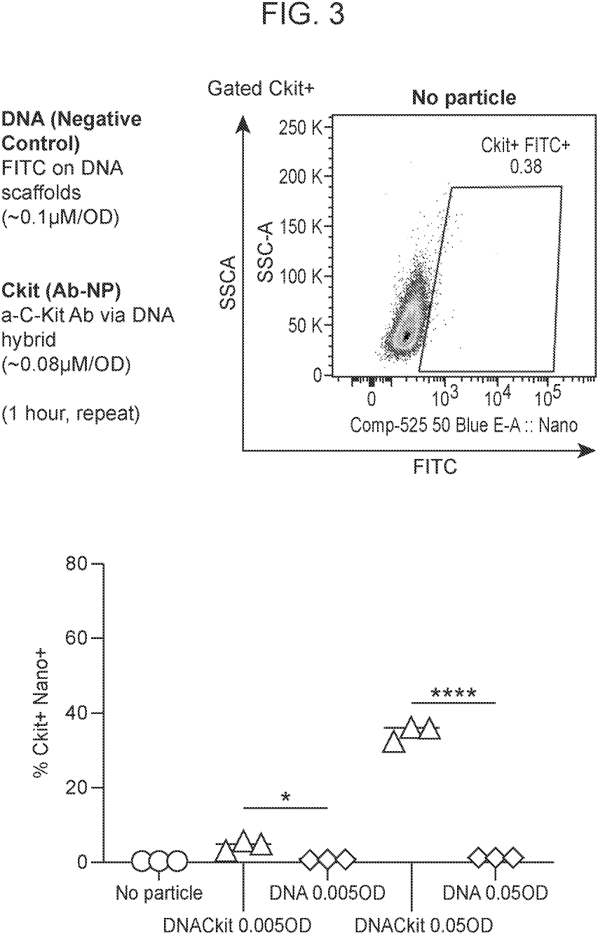

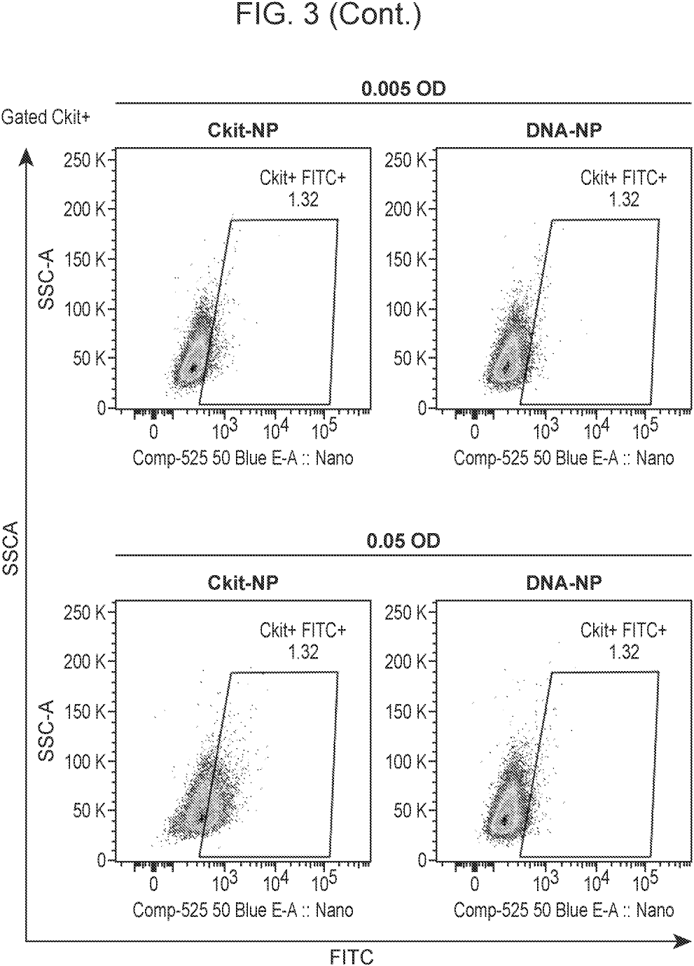

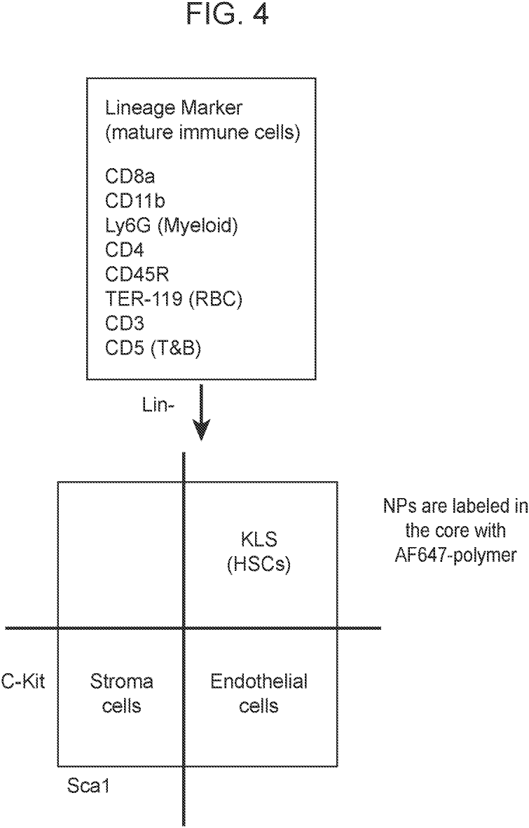

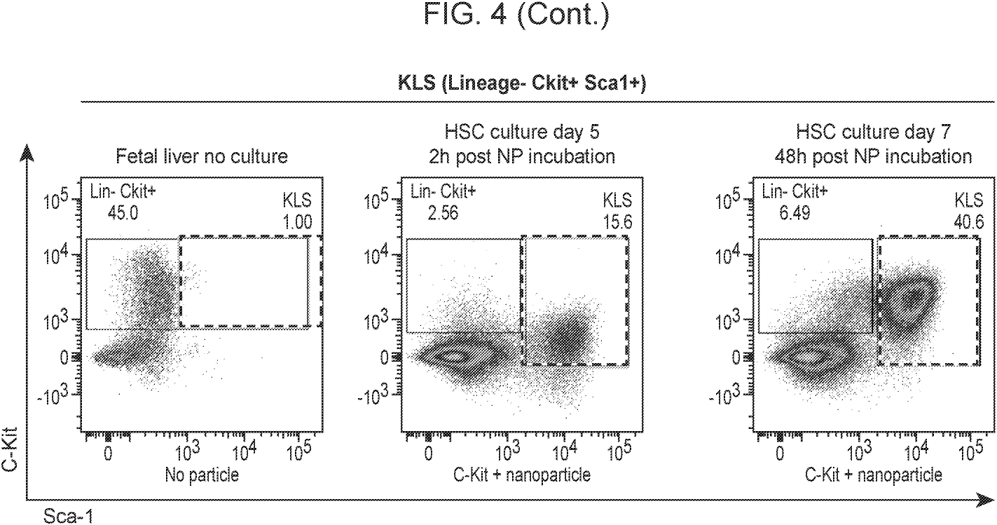

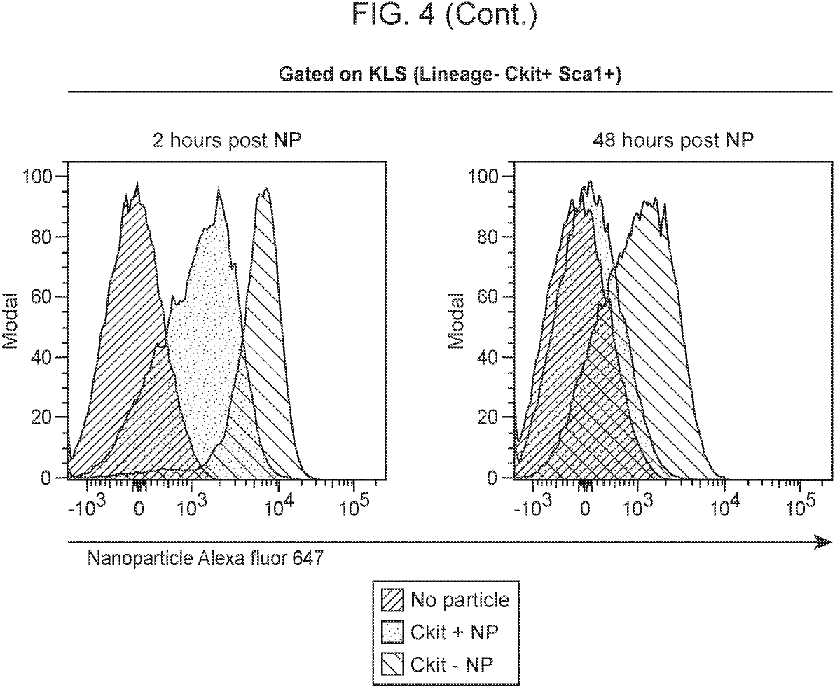

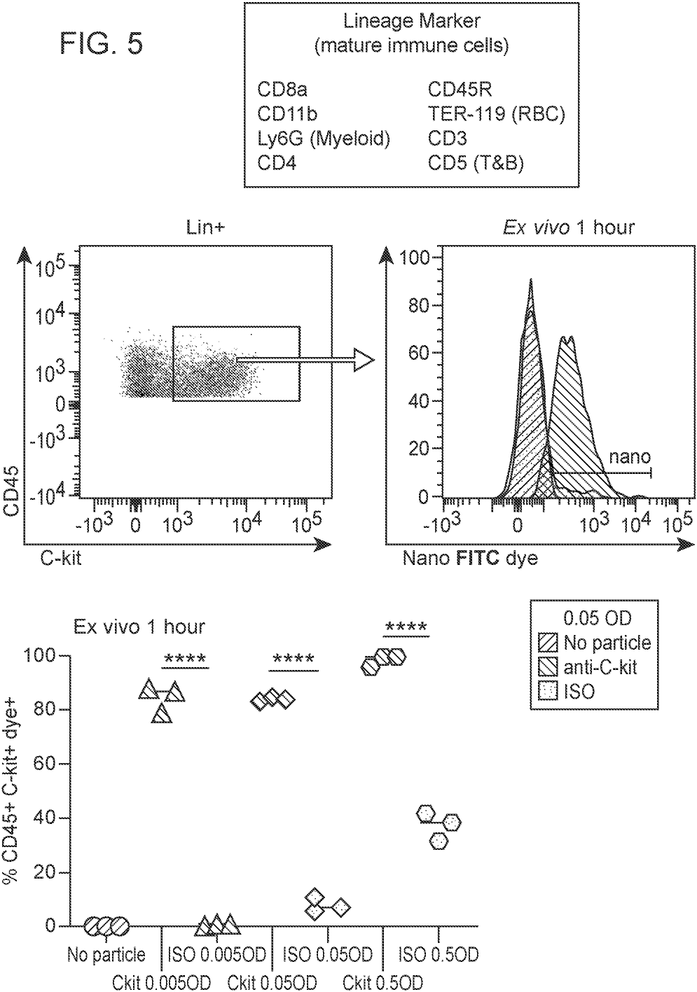

Next, anti-C-kit antibody was conjugated with modified DNA that was complementary to the DNA scaffolds on nanoparticles so that the surface density of the antibody could be precisely controlled. As shown in FIG. 3, nanoparticles coated with anti-C-kit antibody through DNA hybridization showed specific association with C-kit positive cells after ex vivo co-incubation for 1 hour, compared to nanoparticles without the anti-C-kit antibody coating. Murine hematopoietic stem cells (Lin- Ckit+ Sca1+) harvested from fetal livers show higher association with anti-C-kit-coated nanoparticles (AF647 in core) after ex vivo co-incubation, than with nanoparticles without anti-C-kit antibody. It is interesting that C-kit receptor expression level in Sca-1+ cells is decreased at 2 hours post nanoparticle co-incubation, but the expression level is recovered after 48 hours (FIG. 4). Anti-C-kit antibody coated nanoparticles also targeted Lin+ C-kit+ cell subsets isolated from fetal livers, which are differentiation derivatives of HSCs (FIG. 5).

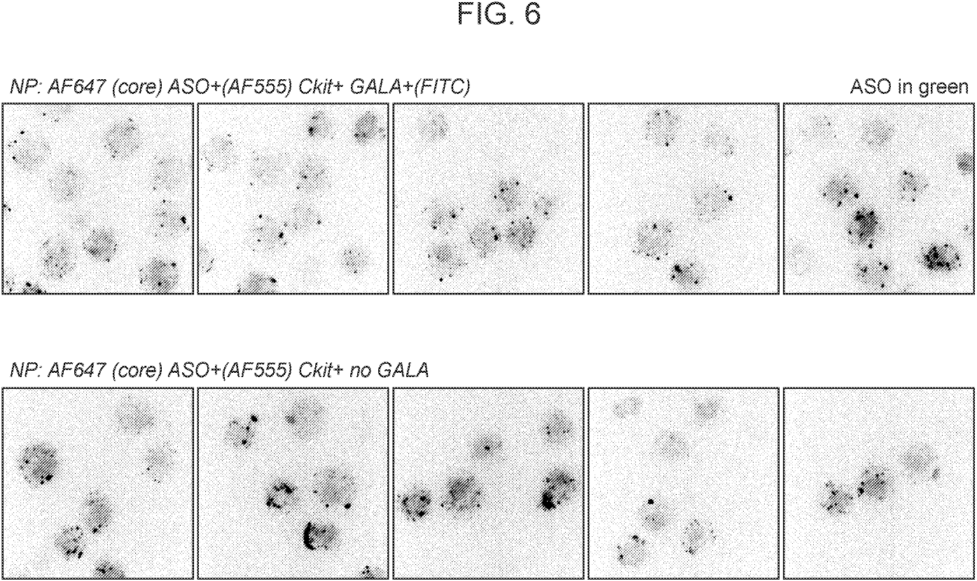

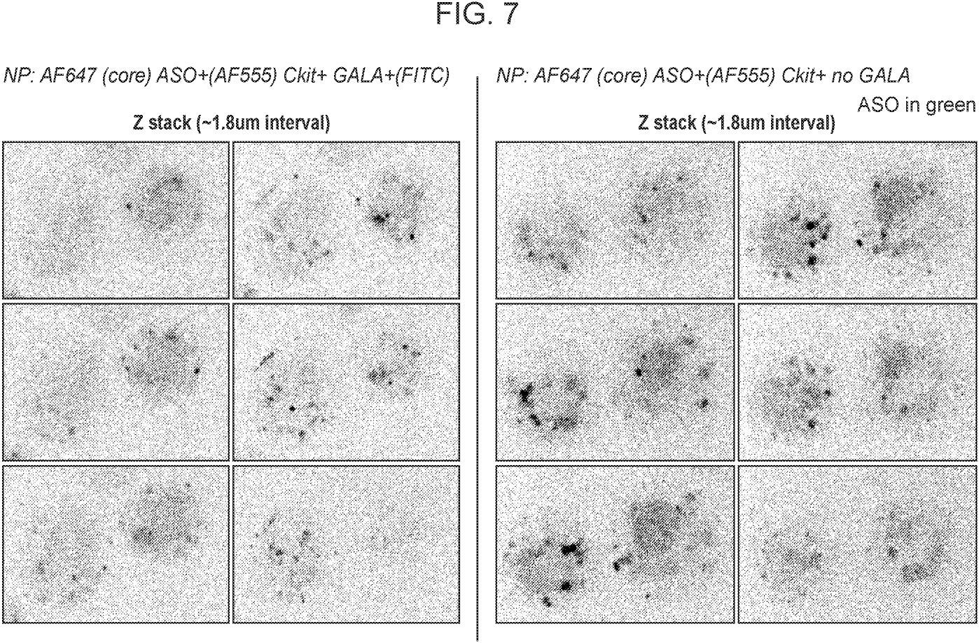

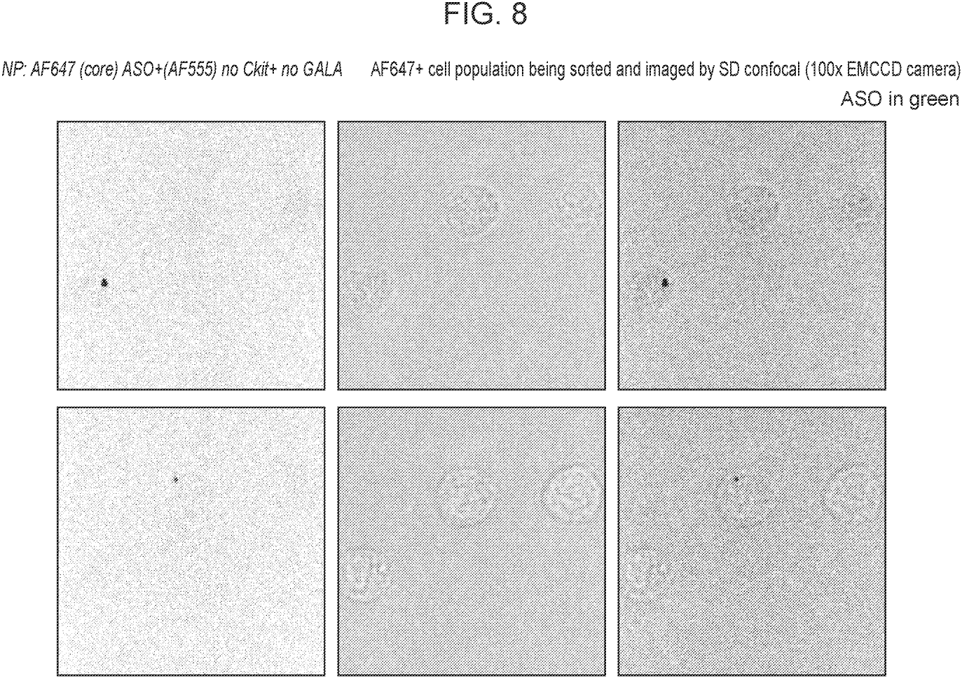

The harvested cells from fetal livers were co-incubated with multifunctional nanoparticles that were coated with anti-C-kit antibody, endosomal escaping peptide GALA, and antisense oligonucleotides (ASOs) for 2 hours. Cells that associated with particles were sorted through a flow sorter, and sorted cells were imaged using a spinning disk confocal microscope at different z-stacks. Images show that nanoparticles are internalized into cells, and GALA peptide coating shows signs of cytosolic release of ASO on nanoparticles (FIGS. 6-8).

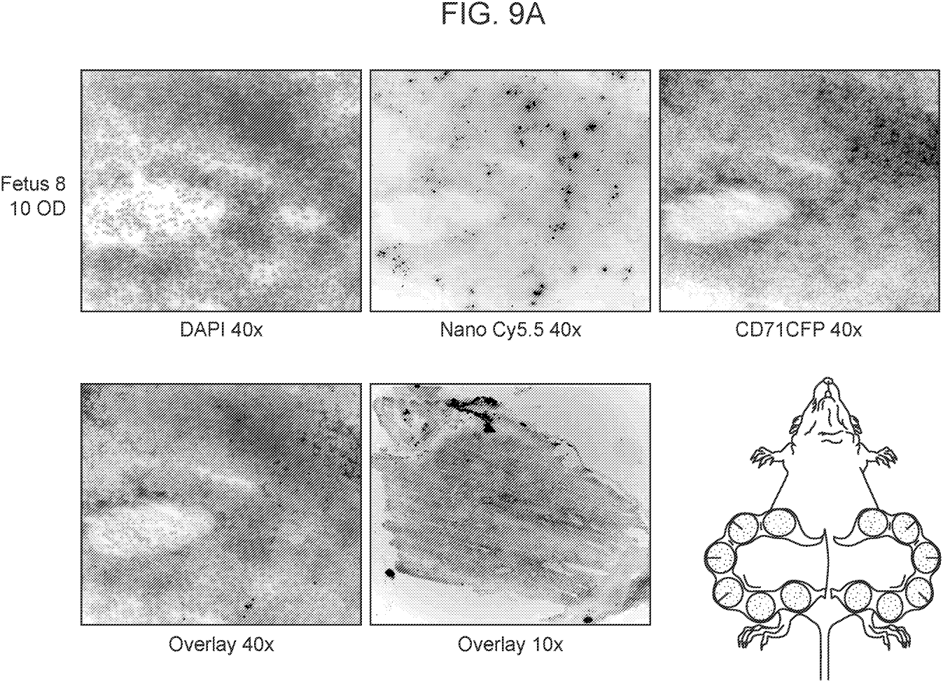

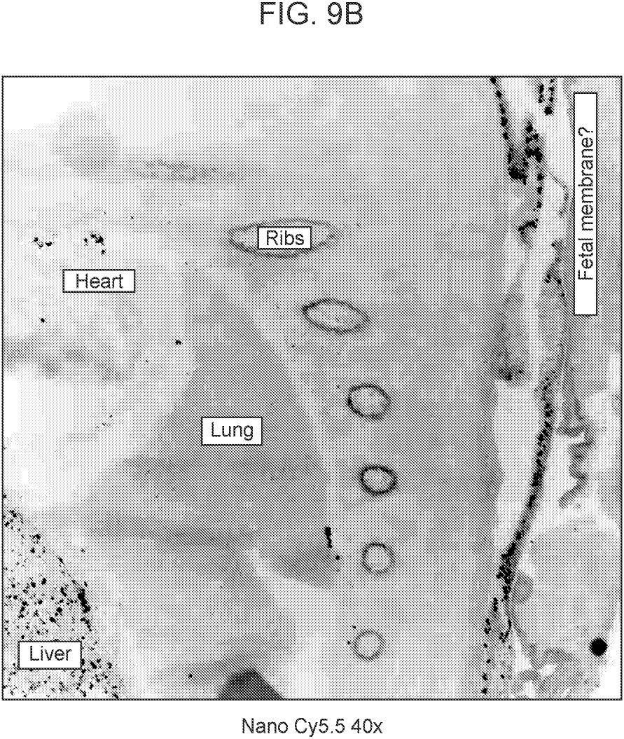

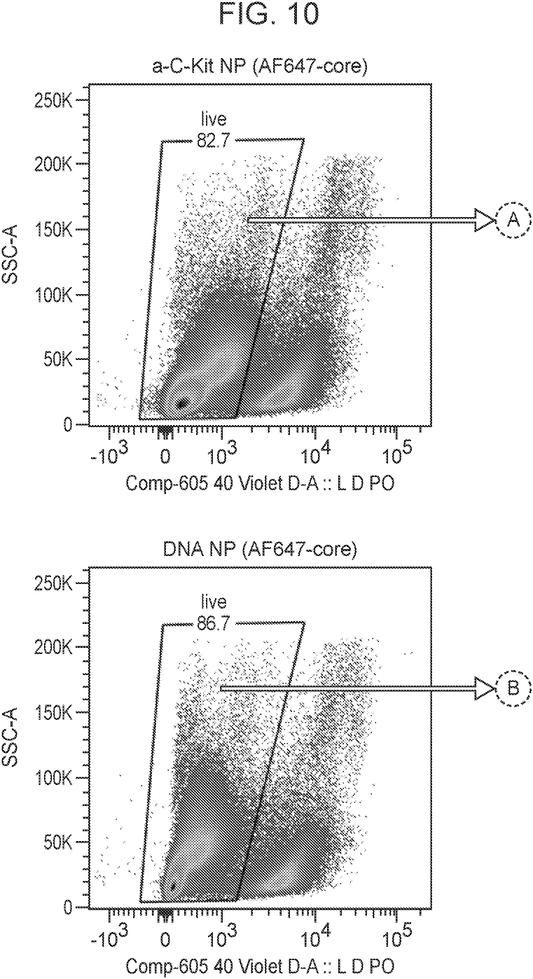

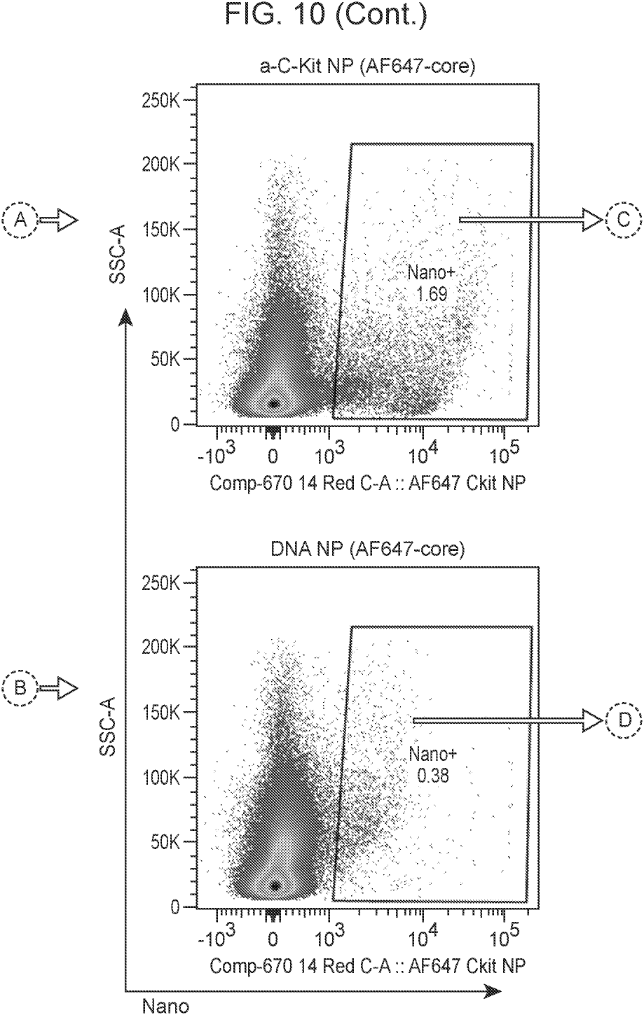

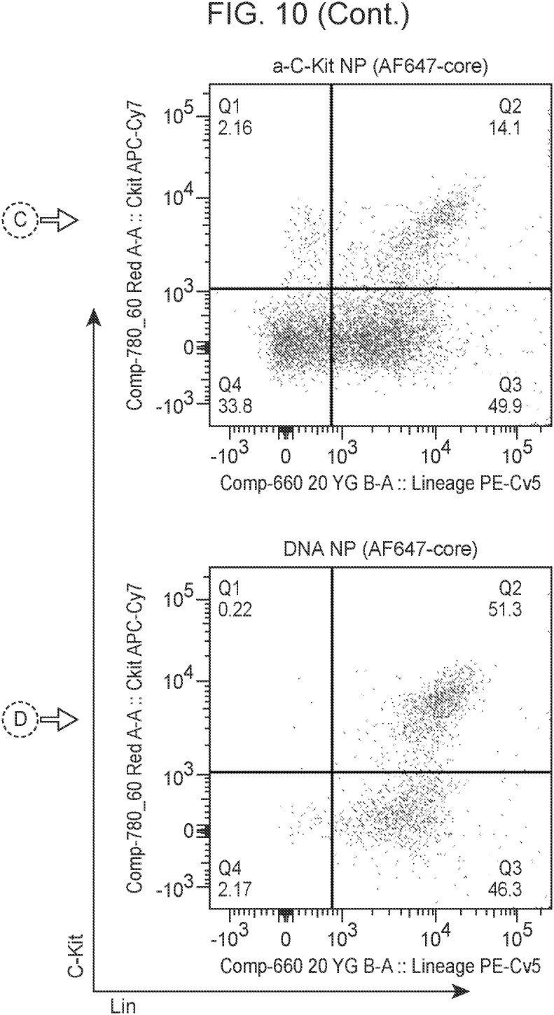

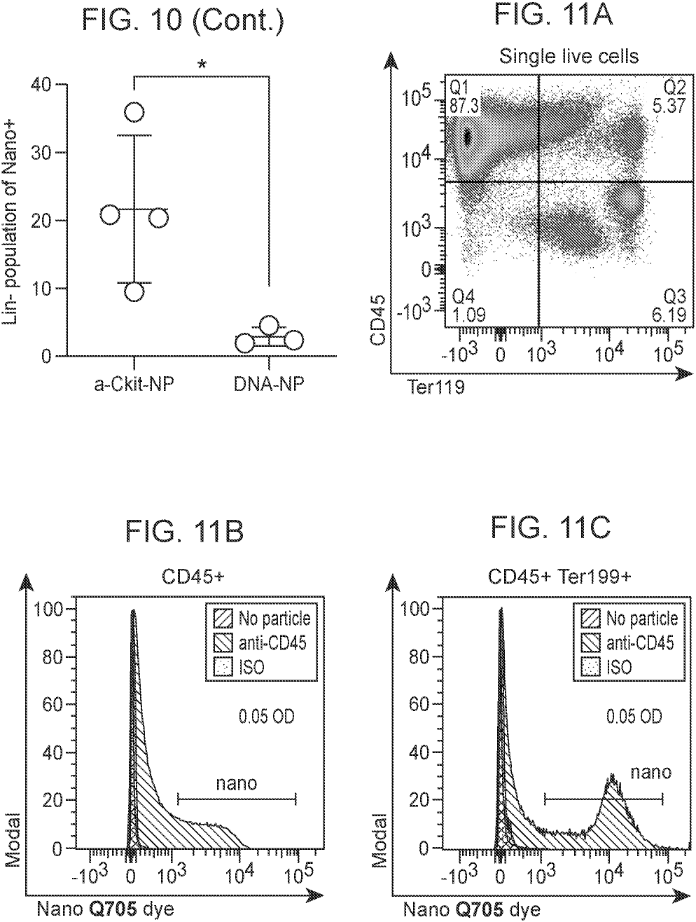

We next compared the biodistribution of nanoparticles coated with anti-C-kit antibody when injected intravenously (IV) or in utero (IU). IVIS imaging was used to determine the organ distribution and immunohistology was performed on the fetus. As shown in FIGS. 9A and 9B, in utero injection of anti-C-kit coated nanoparticles resulted in liver localization in the fetus. In utero injection of nanoparticles that were coated with anti-C-Kit antibodies were internalized more by lineage negative cell subsets than control particles without anti-C-kit antibody after 5 hours (FIG. 10).

### Example 2

### Anti-CD45 Antibody-Coated Nanoparticles for Targeting CD45+ Cell Subsets

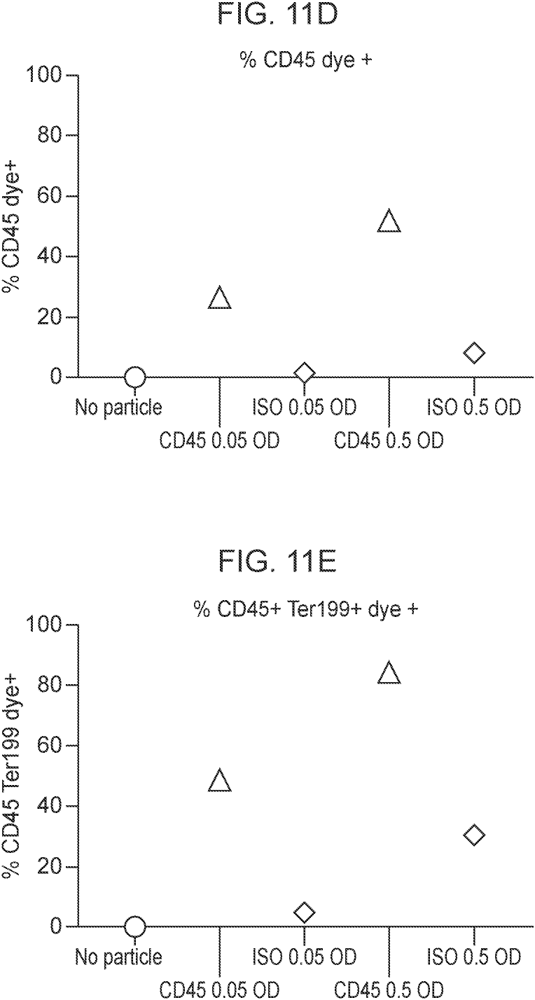

CD45+ cell subsets were targeted using nanoparticles coated with anti-CD45 antibodies. High-density DNA-streptavidin-CD45 - QUasar705 (surface) analysis was performed 1 hour post incubation. The anti-CD45 antibody coated nanoparticles showed specific association with CD45+ cell subsets after 1 hour incubation with harvested cells from the mouse fetal liver (FIGS. 11A-11E).

### Example 3

### Anti- DLK1 Antibody-Coated Nanoparticles for Targeting DLK1+ Cell Subsets

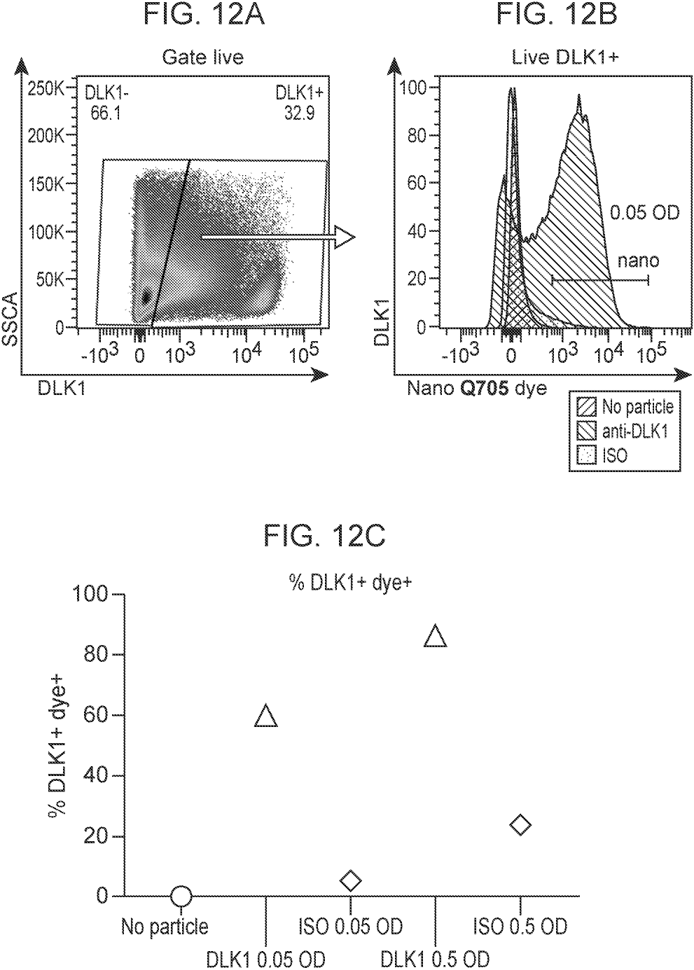

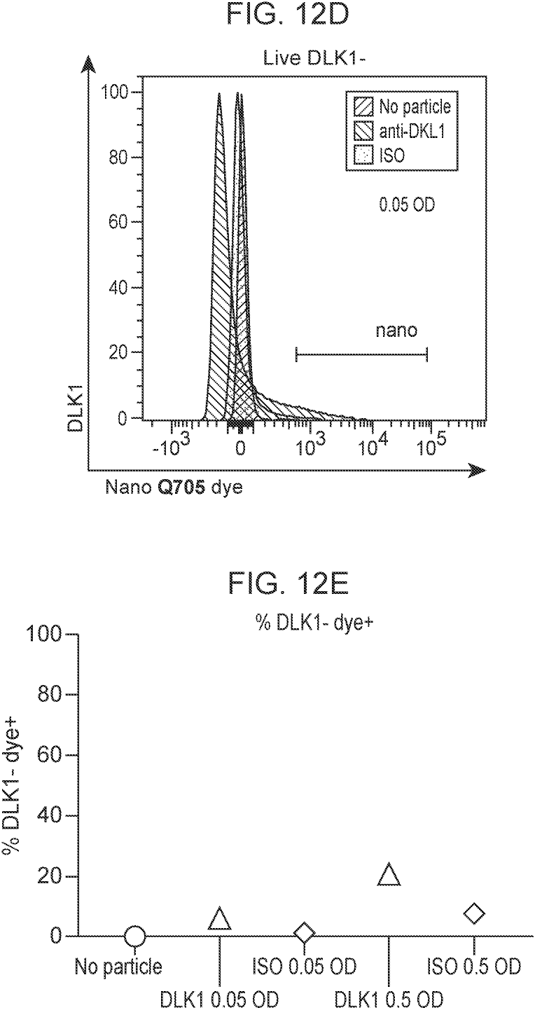

DLK1+ cell subsets were targeted using nanoparticles coated with anti- DLK1 antibodies. FIGS. 12A-12C show the results of high-density DNA-streptavidin-DLK1 - QUasar705 (surface) analysis performed 1 hour post incubation. Anti-DLK1 antibody coated nanoparticles showed specific association with DLK1+ cell subsets after 1 hour incubation with harvested cells from mouse fetal liver.

### Example 4

### CRISPR Knock-In to Add a Sequence to a Gene Via Homology-Directed Repair

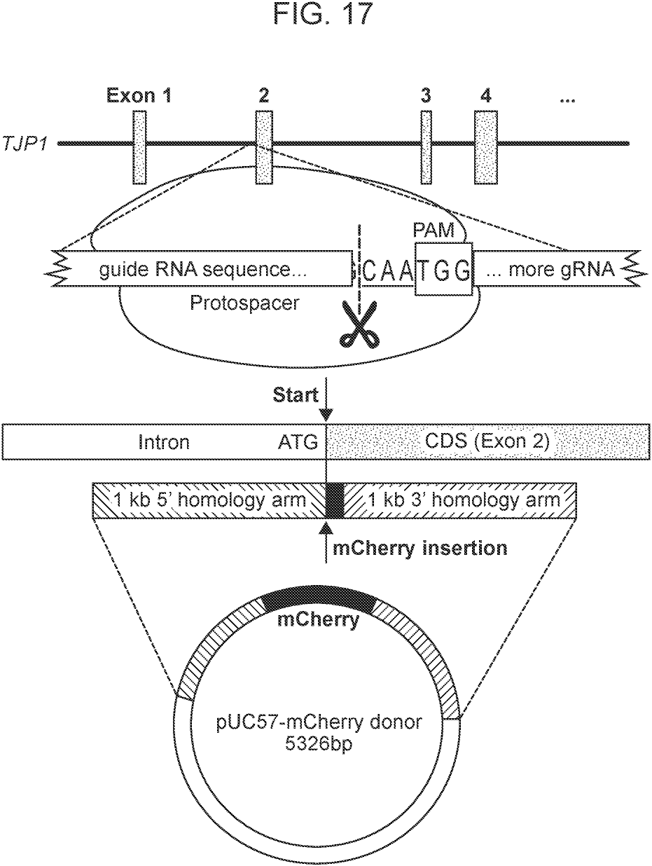

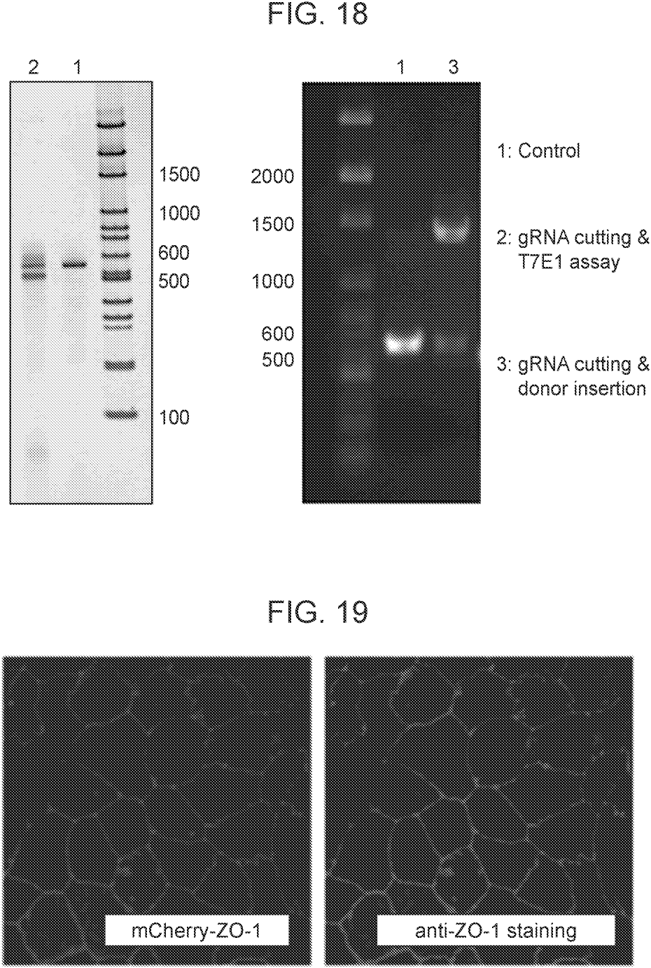

CRISPR was used to add a sequence to a gene via homology directed repair. Specifically, the strategy was employed for CRISPR knock-in of mCherry in Caco-2 cells. A schematic of the guide RNA (gRNA) and donor plasmid design for CRISPR-based knock-in of mCherry to a human TJP1 gene coding for ZO1 protein is shown in FIG. 17. The gRNA cutting site is targeted at exon 2 of TJP1 due to the high GC content of exon 1. An mCherry gene with 1 kb arm at both sides homologous to the up- and down- stream of exon 2 cutting site was synthesized and cloned into the pUC57 plasmid. FIG. 18 shows gels from PCR amplification with primers that flank the cutting site. The gels show efficient Cas9-gRNA based genome cutting and successful mCherry knock-in. On the left is a T7E1 assay post Cas9-gRNA transfection. The original amplicon size was 555 bp and gRNA based cutting yielded fractions of 500 and 55 bp. FIG. 18, at right, shows genomic PCR amplification post Cas9-gRNA and donor plasmid transfection and enrichment by FACS sorting. The amplicon size increased by 750 bp with mCherry insertion. FIG. 19 shows fluorescent images of epithelial Caco-2 cells with mCherry fused at the N-terminus of ZO-1 protein (left) compared to immune-cytostaining of ZO-1 protein of the cells, showing the success of the knock-in strategy. Thus, gene editing reagents may be used to reliably insert in-frame segments into protein coding genes that are still spliced into mRNAs that are transcribed into functional proteins. As shown in the bottom part of FIG. 17, the donor segment included 5′ and 3′ “homology arms”. In certain embodiments, the endogenous homology-directed repair machinery of fetal cells is exploited to repair mutated genes in utero in the fetus. See Wang, 2017, CRISPR/Cas9-based genome editing for disease modeling and therapy: challenges and opportunities for nonviral delivery, Chem Rev. 117:9874-9906 and Liu, 2017, Delivery strategies of the CRISPR-Cas9 gene-editing system for therapeutic applications, J Control Release 266:17-26, incorporated by reference.

Cas9 can be used to knock-in a segment into a genomic protein coding gene in a mammalian system. Homology directed end repair is relied upon in designing a replacement segment to be delivered with the Cas9. A nanoparticle may be used, with the Cas9 and/or the replacement segment either or both packaged in the core or linked to the DNA scaffold embedded in the outer layer of polymer. The replacement segment may include ends that are homologous to a spinal muscle atrophy gene, but that lacks the synonymous single nucleotide difference in exon 7 of SMN2 that otherwise promotes alternative splicing between introns 6 and 8. A composition including the nanoparticles is injected into fetal circulation (e.g., through the umbilical cord). Antibodies against neural precursor cells promote endosomal uptake of the particles into those cells, where the Cas9, guide RNA, and replacement segment are released. The Cas9 inserts the segment into genomic DNA. This results in SMN2 genes that are transcribed to produce SMN pre-mRNA that is spliced into mRNA that will be transcribed into the healthy SMN protein phenotype such that the developing fetus and postnatal patient will avoid the deleterious effects of spinal muscle atrophy.

What is claimed is:

1. A composition for treating a genetic condition, the composition comprising:
a nanoparticle; and
an antisense oligonucleotide carried by the nanoparticle.

2. The composition of claim 1, wherein the antisense oligonucleotide is complementary to, and hybridizes to, a messenger RNA (mRNA) when delivered to a fetal cell.

3. The composition of claim 2, wherein the mRNA is transcribed from a survival motor neuron gene.

4. The composition of any one of claims 1-3, wherein the antisense oligonucleotide is a splice-switching oligonucleotide (SSO).

5. The composition of any one of claims 1-4, wherein the nanoparticle further comprises a plurality of targeting complexes.

6. The composition of claim 4 or 5, wherein the antisense oligonucleotide is an SSO that is complementary to, and hybridizes to, an mRNA from a gene selected from the group consisting of survival motor neuron 1, survival motor neuron 2, β-globin, the IKBKAP gene, UBE3a, genes for other developmental disorders, and the DMD gene.

7. The composition of any one of claims 1-6, wherein the nanoparticle comprises:
an inner region comprising a polymer, a metal, or a liposome; and
an outer region comprising a scaffold of nucleic acid.

8. The composition of claim 7, wherein the antisense oligonucleotide is linked to the nucleic acid of the scaffold.

9. The composition of claim 7 or 8, further comprising one or a plurality of endosomal escape peptides linked to the nucleic acid of the scaffold.

10. The composition of any one of claims 7-9, further comprising a plurality of targeting complexes linked to the nucleic acid of the scaffold.

11. The composition of claim 10, wherein the targeting complexes comprise antibodies that bind to cell-surface markers on fetal cells or cells after birth.

12. The composition of any one of claims 7-11, wherein the antisense oligonucleotide is linked to the nucleic acid of the scaffold by disulfide bonds.

13. The composition of claim 12, further comprising targeting antibodies and/or escape peptides inked to the nucleic acid of the scaffold.

14. The composition of any one of claims 7-13, wherein the polymer comprises poly lactic-co-glycolic acid (PLGA).

15. The composition of any one of claims 7-14, further wherein the inner region of the nanoparticle surrounds a core that contains a payload.

16. The composition of claim 15, wherein the payload comprises one or more of a small molecule, a protein, and a nucleic acid.

17. The composition of any one of claims 12-16, wherein the antisense oligonucleotide is a splice-switching oligonucleotide (SSO) complementary to an mRNA from a survival motor neuron (SMN) gene.

18. The composition of claim 17, wherein, when the nanoparticle is injected into circulation in a fetus:
the targeting complexes target the nanoparticles to neurons or precursors thereof;
the endosomal escape peptides cause release of the nanoparticles into cytosol of the neurons or the precursors thereof;
the SSO is released from the nanoparticle upon exposure to glutathione in the cytosol; and
the SSO binds to an SMN mRNA and prevents formation of an isoform associated with spinal muscle atrophy.

19. The composition of claim 1, wherein an inner region of the nanoparticle comprises a PLGA and an outer region of the nanoparticle comprises a scaffold of nucleic acid linked to the antisense oligonucleotides, targeting complexes, and endosomal escape peptides, wherein the antisense oligonucleotide is a splice-switching oligonucleotide (SSO) complementary to an mRNA from a gene associated with a disease, wherein, when the nanoparticle is injected into circulation in a fetus:
the targeting complexes target the nanoparticles to cells of a specific type;
the escape peptides cause release of the nanoparticles into cytosol of the cells;
the SSO is released from the nanoparticle into the cytosol; and
the SSO binds to an mRNA and prevents formation of splicing of the mRNA into a diseaseassociated isoform.

20. The composition of claim 1, wherein:
the antisense oligonucleotide is between about 10 and 35 nucleotides in length; or
one or more nucleotides in the antisense oligonucleotide includes a modification to prevent degradation by RNase, the modification selected from the group consisting of: base methylation; phosphorothiate (PS) backbone modification; 2′-O-methyl (2′-OMe); 2′-O-methoxyethyl (2′-MOE); Locked nucleic acid (LNA); and phosphorodiamidate morpholinos (PMOs).

21. The composition of claim 1, wherein the gene-editing reagent is targeted to a gene for which a variant promotes an alternative splicing of mRNA that causes a disease, such that when the composition is delivered to a fetus in utero, the gene editing reagent corrects or ameliorates the effect of the variant to thereby inhibit the alternative splicing.

22. The composition of claim 21, wherein the gene-editing reagents, or the nucleic acid encoding the gene-editing reagents, are packaged in a nanoparticle for delivery, wherein an inner region of the nanoparticle comprises a polymer, metal, or liposome, and an outer region of the nanoparticle comprises a scaffold of scaffold nucleic acid.

23. The composition of claim 22, further comprising a plurality of targeting complexes linked to the nanoparticle.

24. The composition of claim 22 or 23, further comprising endosomal escape peptides linked to the nanoparticle.

25. The composition of any one of claims 22-24, wherein the polymer comprises PLGA.

26. The composition of any one of claims 22-25, wherein the gene editing reagents include a zinc-finger nuclease (ZFN), a transcription activator-like effector nuclease (TALEN), or a peptidenucleic acid (PNA).

27. The composition of any one of claims 22-25, wherein the nanoparticle includes the nucleic acids encoding the gene editing reagents and the gene editing reagents include a Cas endonuclease.

28. The composition of any one of claims 22-27, wherein the gene editing reagents include a Cas endonuclease complexed with a guide RNA as a ribonucleoprotein (RNP) within the nanoparticle.

29. The composition of claim 28, wherein the guide RNA targets the Cas endonuclease to a gene to inhibit formation of a splicing isoform of a transcript of the gene.

30. The composition of claim 28, wherein the guide RNA targets the Cas endonuclease to a survival motor neuron gene to inhibit alternative splicing of the SMN gene to inhibit spinal muscle atrophy in the fetus.

31. A therapeutic composition comprising:
a nanoparticle; and
a therapeutic agent carried by or on the nanoparticle.

32. The composition of claim 31, wherein the therapeutic agent comprises a combination of small molecules, nucleotide sequences, and/or proteins.

33. The composition of claim 31 or 32, wherein the therapeutic agent comprises one or more nucleotide sequences.

34. The composition of any one of claims 31-33, wherein the therapeutic agent comprises one or more antisense oligonucleotides (ASO).

35. The composition of claim 34, wherein:
the antisense oligonucleotides are between about 10 and 35 nucleotides in length; and one or more nucleotides in the antisense oligonucleotide includes one or more modifications to prevent degradation, improve RNA binding efficiency, and/or reduce toxicity, the modification selected from the group including: base methylation; phosphorothiate (PS) backbone modification; 2′-O-methyl (2′-OMe); 2′-O-methoxyethyl (2′-MOE); locked nucleic acid (LNA); and phosphorodiamidate morpholinos (PMOs).

36. The composition of any one of claims 31-35, wherein the therapeutic agent comprises one or more splice-switching oligonucleotides (SSO).

37. The composition of any one of claims 31-36, wherein the therapeutic agent comprises one or more short hairpin RNAs (shRNA).

38. The composition of any one of claims 31-37, wherein the therapeutic agent comprises one or more small interfering RNAs (siRNA).

39. The composition of any one of claims 31-38, wherein the therapeutic agent is designed to induce immune tolerance when delivered in utero or after birth.

40. The composition of any one of claims 31-39, wherein the therapeutic agent comprises one or more nucleotide sequences complementary to DNA.

41. The composition of any one of claims 31-40, wherein the therapeutic agent comprises one or more nucleotide sequences complementary to RNA.

42. The composition of any one of claim 31-41, wherein the therapeutic agent comprises one or more nucleotide sequences complementary to the RNA of a gene selected from the group consisting of survival motor neuron 1, survival motor neuron 2, β-globin, blc11a, the IKBKAP gene, the DMD gene, the UBE3A gene, the UBE3A-ATS gene, the SCN2A gene, the SCN8A gene, the SCN3A gene, and genes for other developmental disorders.

43. The composition of any one of claim 31-42, wherein the therapeutic agent comprises one or more nucleotide sequences complementary to the RNA of a gene with multiple isoforms occurring physiologically with the objective of increasing the proportion of transcripts containing specific exons.

44. The composition of any one of claims 31-43, wherein the therapeutic agent comprises one or more nucleotide sequences complementary to the RNA of a gene in which a genetic variant disrupts physiological splicing with the objective of restoring normal splicing behavior.

45. The composition of any one of claims 31-44, wherein the therapeutic agent comprises one or more nucleotide sequences complementary to the RNA of a gene in which a genetic variant leads to an encoded protein that has a gain-of-function or dominant negative effect with the objective of decreasing the quantity of the abnormal RNA or encoded protein.

46. The composition of any one of claims 31-45, wherein the therapeutic agent comprises one or more nucleotide sequences complementary to the genome of a pathogen.

47. The composition of any one of claims 31-46, wherein the nanoparticle further comprises a plurality of targeting complexes.

48. The composition of any one of claims 31-47, further comprising one or a plurality of endosomal escape peptides linked to the nucleic acid of the scaffold.

49. The composition of any one of claims 31-48, further comprising reagents to facilitate passage across the blood-brain barrier.

50. The composition of any one of claims 31-49, wherein the nanoparticle comprises:
an inner region comprising a polymer, a metal, or a liposome; and
an outer region comprising a scaffold of nucleic acid.

51. The composition of claim 50, further comprising a plurality of targeting complexes linked to the nucleic acid of the scaffold.

52. The composition of claim 51, wherein the targeting complexes comprise antibodies that bind to cell-surface markers on fetal cells or cells after birth.

53. The composition of any one of claims 50-52, wherein the polymer comprises poly lactic-co-glycolic acid (PLGA).

54. The composition of any one of claims 50-53, further wherein the inner region of the nanoparticle surrounds a core that contains a payload.

55. The composition of claim 54, wherein the payload comprises one or more of a small molecule, a protein, and a nucleotide sequence.

56. The composition of claim 54 or 55, wherein one or more nucleotide sequences are carried within the polymer or core of the nanoparticle.

57. The composition of any one of claims 50-56, wherein one or more nucleotide sequences are linked to the nucleic acid of the scaffold.

58. The composition of claim 57, wherein the nucleotide sequences are splice-switching oligonucleotides (SSOs) complementary to RNA from a survival motor neuron (SMN) gene.

59. The composition of claim 57, wherein the nucleotide sequences are splice-switching oligonucleotides (SSOs) complementary to RNA from the DMD gene.

60. The composition of claim 57, wherein the nucleotide sequences are antisense oligonucleotides (ASOs) complementary to an antisense RNA or IncRNA including the UBE3A-ATS gene or XIST gene.

61. The composition of any one of claims 50-60, wherein one or more nucleotide sequences are linked to the nucleic acid of the scaffold by disulfide bonds.

62. The composition of any one of claims 50-61, comprising a combination of nucleotide sequences and proteins, including targeting antibodies and/or escape peptides, linked to the nucleic acid of the scaffold.

63. The composition of any one of claims 50-62, comprising a combination of small molecules, nucleotide sequences and proteins, including targeting antibodies and/or escape peptides, linked to the nucleic acid of the scaffold or contained within the polymer or core of the nanoparticle.

64. The composition of any one of claims 50-63, wherein the scaffold of nucleic acid contains multiple nucleotide sequences of varying length and varying degrees of complementarity to a therapeutic nucleotide sequence.

65. The composition of any one of claims 50-64, wherein, when the nanoparticle is injected into circulation before or after birth:
the targeting complexes target the nanoparticles to neurons or precursors thereof;
the endosomal escape peptides cause release of the nanoparticles into cytosol of the neurons or the precursors thereof;
the nucleotide sequences are released from the nanoparticle upon exposure to glutathione in the cytosol; and
the nucleotide sequences bind to RNA to modify splicing or degrade the RNA.

66. The composition of claim 50, wherein an inner region of the nanoparticle comprises a PLGA and an outer region of the nanoparticle comprises a scaffold of nucleic acid linked to nucleotide sequences, targeting complexes, and endosomal escape peptides, wherein the nucleotide sequences are complementary to an RNA from a gene associated with a disease, wherein, when the nanoparticle is injected into circulation in a fetus: the targeting complexes target the nanoparticles to cells of a specific type; the escape peptides cause release of the nanoparticles into cytosol of the cells; the nucleotide sequences are released from the nanoparticle into the cytosol; and the nucleotide sequences bind to RNA to modify splicing or degrade the RNA.

67. The composition of any one of claims 31-66, wherein the therapeutic agent includes splice-switching oligonucleotides (SSO) complementary to an SMN gene RNA and induce the generation of isoforms that produce stable and functional protein to treat spinal muscular atrophy.

68. The composition of any one of claims 31-67, wherein the therapeutic agent includes splice-switching oligonucleotides (SSO) complementary to genes in which a stop codon leads to degradation of the RNA of one or more isoforms with the intent to increase expression.

69. The composition of any one of claims 57-68, in which the nucleotide sequences have a sequence that targets a specific gene and in which: the gene to be targeted is DMD to skip exons with genetic variants leading to muscular dystrophy; or the gene to be targeted is SCN1A to skip exons that would lead to nonsense-mediated decay as a treatment for Dravet syndrome.

70. The composition of any one of claims 57-68, wherein the nucleotide sequences are antisense oligonucleotides (ASOs) complementary to the UBE3A-ATS RNA leading to its degradation resulting in upregulation of the UBE3A gene to treat Angelman syndrome.

71. A composition comprising:
gene-editing reagents, or a nucleic acid comprising nucleotide sequences encoding the gene-editing reagents, wherein the gene-editing reagents are targeted to a gene for which a variant contributes to a disease, such that when the composition is delivered to a fetus in utero, the gene editing reagents correct or ameliorate the effect of the variant; or
gene-editing reagents, or nucleotide sequences encoding the gene-editing reagents, wherein the gene-editing reagents are targeted to a gene that modifies a disease process, such that when the composition is delivered to a fetus in utero, the gene editing reagents correct or ameliorate the disease process; or
reagents targeted to the cis regulatory regions of a gene, or nucleotide sequences encoding the reagents, wherein the reagents are targeted to a gene for which a variant causes a disease process, such that when the composition is delivered to a fetus in utero, the reagents correct or ameliorate the disease process by increasing gene expression, decreasing gene expression or modifying splicing and isoform usage.

72. The composition of claim 71, wherein the reagents, or the nucleotide sequences encoding the reagents, are packaged with a nanoparticle for delivery.

73. The composition of claim 72, wherein the nanoparticle comprises:
an inner region comprising a polymer, a metal, or a liposome; and
an outer region comprising a scaffold of nucleic acid.

74. The composition of claim 73, wherein the polymer comprises PLGA.

75. The composition of any one of claims 72-74, further comprising a plurality of targeting complexes linked to the nanoparticle.

76. The composition of any one of claims 72-75, further comprising endosomal escape peptides linked to the nanoparticle.

77. The composition of any one of claims 72-76, wherein the gene editing reagents include a zinc-finger nuclease (ZFN), a transcription activator-like effector nuclease (TALEN), a peptidenucleic acid (PNA), or a Cas endonuclease.

78. The composition of any one of claims 72-77, wherein the nanoparticle includes nucleotide sequences encoding the gene editing reagents and the gene editing reagents include a Cas endonuclease.

79. The composition of any one of claims 72-78, wherein the gene editing reagents include a Cas endonuclease complexed with a guide RNA as a ribonucleoprotein (RNP) within the nanoparticle.

80. The composition of claim 79, wherein the guide RNA targets the Cas endonuclease to a survival motor neuron gene to modify splicing of the SMN gene to produce stable and functional SMN protein to treat spinal muscle atrophy in the fetus.

81. The composition of claim 79, wherein the guide RNA targets the Cas endonuclease to the CFTR gene to produce stable and functional CFTR protein to treat cystic fibrosis in the fetus.

82. The composition of any one of claims 72-81, wherein the nanoparticle includes nucleotide sequences encoding a gene to replace one or more copies that are defective leading to disease in an individual.

83. The composition of claim 82, wherein the gene to be replaced is: CFTR, HBB, SERPINA1, SLC26A4, KCNJ10, GALNS, DMD, F8, F9, F9, HBA2, HBA1, FMR1, HGSNAT, SFTPB, SGSH, SMN1, GBA, or SCARB2.

84. A composition comprising:
a nanoparticle;
a payload carried by the nanoparticle; and
one or more targeting complexes linked to the nanoparticle.

85. The composition of claim 84, wherein the nanoparticle comprises:
an inner region comprising a polymer, a metal, or a liposome; and
an outer region comprising a scaffold of nucleic acid.

86. The composition of claim 85, wherein the payload is linked to the nucleic acid of the scaffold.

87. The composition of claim 85 or 86, further comprising one or more endosomal escape peptides linked to the nucleic acid of the scaffold.

88. The composition of any one of claims 84-87, wherein the targeting complexes comprise antibodies that bind to cell-surface markers on stem cells.

89. The composition of claim 88, wherein the antibodies comprise α-c-kit antibodies.

90. The composition of any one of claims 84-89, wherein the nanoparticle has a coating comprising antibodies targeting c-kit⁺ cells.

91. The composition of any one of claims 85-90, wherein the polymer comprises poly lactic-co-glycolic acid (PLGA).

92. The composition of any one of claims 85-91, wherein the inner region of the nanoparticle surrounds a core that contains a payload.

93. The composition of any one of claims 84-92, wherein the payload comprises one or more of a small molecule, a protein, and a nucleic acid.

94. The composition of any one of claims 84-93, wherein the payload comprises gene editing reagents or nucleic acids encoding the gene editing reagents.

95. The composition of claim 94, wherein the gene editing reagents include at least one cas9 endonuclease and a guide RNA.

96. The composition of claim 95, wherein the payload includes a mRNA, a plasmid, or a viral vector encoding at least one cas9 endonuclease and/or a guide RNA.

97. A method comprising delivering a composition according to any one of claims of 84-96 to stem cells to introduce the payload into the stem cells.

98. The method of claim 97, wherein the stem cells are hematopoietic stem cells (HSCs).

99. The method of claim 97 or 98, wherein the delivery is performed in vitro or in vivo.

100. The method of any one of claims 97-99, wherein the nanoparticle comprises:
an inner region comprising a polymer;
an outer region comprising a scaffold of nucleic acid; and
a coating of anti C-kit antibodies.

101. The method of claim 100, wherein the polymer is poly lactic-co-glycolic acid (PLGA).
